SEQUENCE LISTING (11 sequences)

SEQ ID NO: 1 - protein, 26 aa, Artificial Sequence
  synthetic amphipathic alpha helix HDL mimetic peptide resistant to oxidation that stimulates ATP binding cassette protein ABCA1-dependent cholesterol efflux based on apolipoprotein AI (ApoA-I) alpha-helical motif
XXXXRXXXXXXXXXXXXXXXXXXXRX

SEQ ID NO: 2 - protein, 26 aa, Artificial Sequence
  synthetic amphipathic alpha helix HDL mimetic peptide resistant to oxidation that stimulates ATP binding cassette protein ABCA1-dependent cholesterol efflux based on apolipoprotein AI (ApoA-I) alpha-helical motif
EXRSRLEEXFAAFREFAEEFLARLRS

SEQ ID NO: 3 - protein, 26 aa, Artificial Sequence
  synthetic amphipathic alpha helix HDL mimetic peptide resistant to oxidation that stimulates ATP binding cassette protein ABCA1-dependent cholesterol efflux based on apolipoprotein AI (ApoA-I) alpha-helical motif
EVRSRLEEXFAAFREFAEEFLARLRS

SEQ ID NO: 4 - protein, 26 aa, Artificial Sequence
  synthetic amphipathic alpha helix HDL mimetic peptide ATI-5261 susceptible to oxidation that stimulates ATP binding cassette protein ABCA1-dependent cholesterol efflux based on apolipoprotein AI (ApoA-I) alpha-helical motif
EVRSKLEEWFAAFREFAEEFLARLKS

SEQ ID NO: 5 - protein, 6 aa, Artificial Sequence
  synthetic Factor Xa cleavage site
MHIEGR

SEQ ID NO: 6 - protein, 26 aa, Artificial Sequence
  synthetic amphipathic alpha helix HDL mimetic peptide ATI-5261 analog having Arg substituted for Lys at positions 5 and 25 based on apolipoprotein AI (ApoA-I) alpha- helical motif, peptide K5,25->R analog of ATI-5261
EVRSRLEEWFAAFREFAEEFLARLRS

SEQ ID NO: 7 - protein, 26 aa, Artificial Sequence
  synthetic amphipathic alpha helix HDL mimetic peptide ATI-5261 analog having Arg substituted for Lys at positions 5 and 25 and Trp changed to Leu at position 9 based on apolipoprotein AI (ApoA-I) alpha-helical motif, peptide K5,25->R, W9->L analog of ATI-5261
EVRSRLEELFAAFREFAEEFLARLRS

SEQ ID NO: 8 - protein, 26 aa, Artificial Sequence
  synthetic amphipathic alpha helix HDL mimetic oxidation-susceptible (Ox-S) form of ATI-5261 peptide that stimulates ATP binding cassette protein ABCA1-dependent cholesterol efflux
EVRSKLEEWFAAFREFAEEFYARLKS

SEQ ID NO: 9 - protein, 26 aa, Artificial Sequence
  synthetic amphipathic alpha helix HDL mimetic oxidation-resistant (Ox-R) form of ATI-5261 peptide that stimulates ATP binding cassette protein ABCA1-dependent cholesterol efflux
EVRSRLEELFAAFREFAEEFYARLRS

SEQ ID NO: 10 - protein, 26 aa, Artificial Sequence
  synthetic amphipathic alpha helix HDL mimetic oxidation-susceptible (Ox-S) form of ATI-5261 peptide with V2->L substitution that stimulates ATP binding cassette protein ABCA1-dependent cholesterol efflux
ELRSKLEEWFAAFREFAEEFYARLKS

SEQ ID NO: 11 - protein, 27 aa, Artificial Sequence
  synthetic amphipathic alpha helix HDL mimetic oxidation-resistant (Ox-R) form of ATI-5261 peptide with V2->L substitution that stimulates ATP binding cassette protein ABCA1-dependent cholesterol efflux
ELVRSRLEELFAAFREFAEEFYARLRS

(19) United States
(12) Patent Application Publication
(10) Pub. No.: US 2013/0123173 A1
(43) Pub. Date: May 16, 2013

(54) CREATION OF OXIDATION-RESISTANT HDL MIMETIC PEPTIDES

(71) Applicants: John K. Bielicki, San Ramon, CA (US); Jan Johansson, San Ramon, CA (US)

(72) Inventors: John K. Bielicki, San Ramon, CA (US); Jan Johansson, San Ramon, CA (US)

(73) Assignee: The Regents of the University of California, Oakland, CA (US)

(21) Appl. No.: 13/643,935

(22) PCT Filed: Apr. 28, 2011
(86) PCT No.: PCT/US11/34279

§ 371 Date: Jan. 9, 2013

Related U.S. Application Data
(60) Provisional application No. 61/329,049, filed on Apr. 28, 2010.

(51) Int. Cl. C07K 14/00; A61K 38/16

(52) U.S. Cl. CPC C07K 14/001; A61K 38/16
USPC 514/7.4; 530/333; 530/300

(57) ABSTRACT

The present invention provides non-naturally occurring polypeptides that are oxidation resistant and have cholesterol efflux activity that parallels that of full-length apolipoproteins (e.g., Apo AI and Apo E), and having high selectivity for ABACI that parallels that of full-length apolipoproteins. The invention also provides compositions comprising such polypeptides, methods of identifying, screening and synthesizing such polypeptides, methods of treating, preventing or diagnosing diseases and disorders associated with dyslipidemia, hypercholesterolemia and inflammation; and diagnostic methods employing the peptides to evaluated cholesterol efflux activity.

### CROSS-REFERENCE TO RELATED APPLICATION

This application claims benefit of U.S. provisional application no. 61/329,049, filed Apr. 28, 2010, which application is incorporated by reference herein for all purposes.

### STATEMENT AS TO RIGHTS TO INVENTIONS MADE UNDER FEDERALLY SPONSORED RESEARCH AND DEVELOPMENT

This invention was made with government support under Contract No. DE-AC02-05CH11231 awarded by the U.S. Department of Energy and Grant (Contract) No. R03-AG023153 awarded by the National Institutes of Aging. The Government may have certain rights in this invention.

The research leading to this invention was also funded by a sponsored research agreement with Artery Therapeutices, Inc. (LBNL Work for Agreement No. LB05-001119) and by Grant No. 13IT-0025 and 17RT-0082 awarded by the Tobacco Related Disease Research Program of the State of California.

### BACKGROUND OF THE INVENTION

Atherosclerosis is a common form of cardiovascular disease and leading cause of morbidity and mortality. Modifying risk factors through life-style changes and lipid-lowering medicines represents current standard of care. These efforts reduce coronary events by only 30%, indicating new therapeutic strategies are needed to fulfill unmet medical needs.

Strategies centered on plasma HDL hold promise in the treatment of atherosclerosis. Epidemiological and clinical studies indicate that HDL and its major protein apoA-I directly protect against atherosclerosis. This can be attributed, in part, to activity of HDL/apoA-I to remove excess cholesterol from cells of arterial-plaque (cholesterol efflux activity) and to transport cholesterol to the liver for excretion in feces (reverse cholesterol transport). Current treatments based on HDL often employ full-length recombinant forms of apoA-I complexed with phospholipids. These types of therapies are cumbersome and costly to produce and raise safety concerns associated with immunological responses.

An alternative to the use of full-length proteins involves small peptides based on α-helical motifs of apoA-I (see, e.g., WO 2008/115303; W02009155366). For example, a 26-mer peptide (ATI-5261) stimulates cellular cholesterol efflux via the membrane protein ABCA1, present on macrophage-foam cells in atherosclerotic plaque. Moreover, the peptide displays nearly the same molar potency as native apoA-I. This peptide was also found to reduce substantial atherosclerosis in mice fed high-fat western-diet and, thus, may be useful for the treatment of atherosclerosis.

Oxidative events occurring during the development of atherosclerosis in humans, including those involving myeloperoxidase (MPO), are thought to impede HDL activity by damaging the apoA-I protein and preventing or decreasing its ability to mediate ABCA1 cholesterol efflux. Not to be bound by theory, this is thought to involve oxidative products that form adducts with apoA-I amino acids, thereby inhibiting the apoA-I interaction with ABCA1.

The present invention relates to the development of oxidation-resistant peptides for the study, treatment, and imaging of atherosclerotic plaque.

### SUMMARY OF THE INVENTION

Peptides of the invention that are resistant to oxidation are typically designed with arginine residues (principal cationic amino acid) instead of lysine residues and/or leucine (or phenylalanine) residues instead of tryptophan residues. The oxidation-resistant peptides have great potential to reduce atherosclerosis in humans, by virtue of their ability to remain active and mediate ABCA1 cholesterol efflux and/or promote pre-β HDL formation during oxidative events in the artery wall. Such peptides may also be useful for imaging developing or vulnerable atherosclerotic plaque, as they retain targeting ability and bind ABCA1 in the presence of inflammation/oxidative stress.

Thus, in one aspect, the invention provides a method of making peptides that are oxidation resistant, the method comprising synthesizing a peptide of less than about 100 amino acids in length, e.g., of 60 amino acids or less in length, wherein the peptide comprises an amphipathic alpha helix of 18 to 40 amino acids, e.g., 20, 24, 26, 28, 30, 32, 34, 36, 38, or 40, amino acids in length, wherein the polar face comprises acidic amino acids positioned within the alpha helix structure, e.g., positioned approximately evenly (e.g., at about every one, two or three helical turns); and wherein a positively charged residue at the lipid/water interface of the amphipathic alpha helix is arginine. An oxidation resistant peptide is typically synthesized without any of the following residues: tryptophan, tyrosine, cysteine, methionine, or lysine.

In some embodiments, a peptide of the invention is synthesized in accordance with the following characteristics of the amphipathic alpha helix: X₁X₂X₃X₄RX₆X₇X₈(L/F)X₁₀X₁₁X₁₂X₁₃X₁₄X₁₅X₁₆X₁₇X₁₈X₁₉X₂₀X₂₁X₂₂X₂₃X₂₄ RX₂₆ (SEQ ID NO:1), wherein: X₁, X₇, X₈, X₁₅, X₁₈ and X₁₉ are amino acids independently selected from the group consisting of E and D; X₂, X₆, X₁₀, X₁₂, X₁₃, X₁₆, X₁₇, X₂₀, X₂₁ and X₂₄ are amino acids independently selected from the group consisting of A, V, L, I, or F; X₃, X₁₄ and X₂₃ are amino acids independently selected from the group consisting of R, A, V, L, I, F, G, S, T, N and Q, wherein at least two of X₃, X₁₄ and X₂₃ are R; and X₄, X₁₁, X₂₂ and X₂₆ are amino acids independently selected from the group consisting of T, S, G, and A. In some embodiments, X₂, X₆, X₁₀, X₁₂, X₁₃, X₁₆, X₁₇, X₂₀, X₂₁ and X₂₄ are amino acids independently selected from the group consisting of A, V, I, L, and F. In some embodiments, X₂ is V or L. In some embodiments, X₂, X₆, X₁₀, X₁₂, X₁₃, X₁₆, X₁₇, X₂₀, X₂₁ and X₂₄ are amino acids independently selected from the group consisting of L and F. In some embodiments, X₄, X₁₁, and X₂₂ are A.

In some embodiments, the peptide is synthesized in accordance with the following: the amphipathic alpha helix comprises at least 80% identity, or at least 90% identity, or at least 95% identity, to E(V/L)RSRLEE(L/F)FAA FREFAEEFLARLRS (SEQ ID NO:2), the residues at positions 5 and 25 are R; and the residue at position 9 is L or F. In some embodiments, the amphipathic alpha helix comprises the sequence EVRSRLEE(L/F)FAAFREFAEEFLARLRS (SEQ ID NO:3). In some embodiments, the residue at position 9 is L.

The invention also provides oxidation resistant peptides synthesized in accordance with the methods of the invention. In some embodiments, a peptide of the invention has increased activity relative to an oxidation sensitive counterpart peptide that comprises an amphipathic alpha helix that has one or more lysine residues at the lipid/water interface and comprises one or more tryptophan or tyrosine residues, e.g., a tryptophan at a position that is L or F in the oxidation resistant peptide. In some embodiments, an oxidation-resistant peptide of the invention has increased activity in the presence of an agent such as an acrolein, in comparison to when the agent is not present. Acrolein is a product of lipid peroxidation in vivo; that is, it can be formed from decomposition of lipid, which results from oxidative damage (enzymatic or non-enzymatic). Acrolein can also be generated from products of the enzyme myeloperoxidase (MPO) damaging protein threonine residues. Thus, acrolein is example of aldehyde that can be generated in vivo by both lipid and protein oxidation.

In another aspect, the invention also provides peptides that are developed with oxidation susceptible residues and motifs to obtain oxidation-resistant and -susceptible peptides of nearly identical composition and structure for quantifying differential reverse cholesterol transport (RCT) responses, thus providing a means for identifying subjects at greater risk for developing atherosclerosis and/or assessing HDL dysfunction. Identification of such subjects is useful for devising biomarkers, identifying drug candidates or patient responders, assessing efficacy of therapeutic interventions, and/or eludicating disease mechanisms.

The invention thus relates to HDL mimetic oxidation resistant peptides that a) stimulate ABCA1 cholesterol efflux with apoA-I molar potency and b) greatly reduce established atherosclerosis in hypercholesterolemic mice. A prototype peptide, ATI-5261, was used to identify amino acids that confer efflux activity, for the purpose of engineering oxidation resistant analogs. Thus, the invention is based, in part, on the discovery that Lysine to Arginine (K→R) substitutions at positions 5 and 25 in ATI-5261 had no effect on cholesterol efflux activity (in absence of oxidants), but exhibited greatly improved macrophage reverse cholesterol transport upon exposure to oxidative stress. Moreover, the invention provides highly potent ABCA1 efflux peptides of nearly identical composition, that can display markedly different susceptibility to MPO oxidants. Such peptides are useful for developing biomarkers and strategies to optimize efficacy of HDL therapies.

Peptides of the current invention possess unprecedented activity for mediating ABCA1 cholesterol efflux and thus are targeting platforms for devising novel therapies and imaging agents for cardiovascular disease. Moreover, the present structural themes allow the development of novel compounds resistant to oxidation products/stress, ensuring effective targeting performance to optimize therapeutic efficacy/disease assessment and imaging.

In some embodiments, the invention provides a kit comprising an oxidation resistant polypeptide and an oxidation susceptible counterpart polypeptide wherein the oxidation-resistant polypeptide comprises an amphipathic alpha helix having the following characteristics X₁X₂X₃X₄RX₆X₇X₈(L/F)X₁₀X₁₁X₁₂X₁₃X₁₄X₁₅X₁₆X₁₇X₁₈X₁₉X₂₀X₂₁X₂₂X₂₃X₂₄RX₂₆ (SEQ ID NO:1), wherein: X₁, X₇, X₈, X₁₅, X₁₈ and X₁₉ are amino acids independently selected from the group consisting of E and D; X₂, X₆, X₁₀, X₁₂, X₁₃, X₁₆, X₁₇, X₂₀, X₂₁ and X₂₄ are amino acids independently selected from the group consisting of A, V, L, I, F, or M; X₃, X₁₄ and X₂₃ are amino acids independently selected from the group consisting of R, A, V, L, I, F, M, G, S, T, N and Q, wherein at least two of X₃, X₁₄ and X₂₃ are R; and X₄, X₁₁, X₂₂ and X₂₆ are amino acids independently selected from the group consisting of T, S, G, and A. In some embodiments, X₂, X₆, X₁₀, X₁₂, X₁₃, X₁₆, X₁₇, X₂₀, X₂₁ and X₂₄ are amino acids independently selected from the group consisting of A, V, L, and F. In some embodiments, X₂ is V or L. In some embodiments, X₂, X₆, X₁₀, X₁₂, X₁₃, X₁₆, X₁₇, X₂₀, X₂₁ and X₂₄ are amino acids independently selected from the group consisting of L and F. In some embodiments, X₄, X₁₁, and X₂₂ are A. The counterpart oxidation susceptible polypeptide included in the kit typically includes a K at positions 5 and 25 and further, may include a W at position 9 and/or a Y at position 21. In some embodiments, the residue at position 9 is L.

In some embodiments, the oxidation-resistant polypeptide comprises an amphipathic alpha helix that has at least 80% identity, or at least 90% identity, or at least 95% identity, to E(V/L)RSRLEE(L/F)FAAFREFAEEFLARLRS (SEQ ID NO:2), the residue at position 5 and 25 is R; and the residue at position 9 is L or F. In some embodiments, the amphipathic alpha helix comprises the sequence EVRSRLEE(L/F)FAAFREFAEEFLARLRS (SEQ ID NO:3). An oxidation susceptible counterpart polypeptide of such a peptide comprises K at positions 5 and 25 and in some embodiments, comprises a W at position 9 and/or a Y at position 21. In some embodiments, the residue at position 9 is L.

The present invention thus provides additional practical and efficacious alternatives to use of full-length proteins and reconstituted HDL nanodiscs, currently being developed for clinical use as therapies and imaging agents. The oxidation-resistant peptide forms retain activity and ability to bind ABCA1 in presence of oxidative events and, therefore, represent an effective means for targeting ABCA1 during atherosclerosis. This characteristic of the peptides of the invention is in contrast to native apoA-I, which loses activity toward ABCA1 upon exposure to oxidants.

Differential cholesterol efflux/reverse cholesterol transport responses between oxidation-resistant and -susceptible peptides represents a valuable means for assessing in vivo RCT, disease mechanisms, and efficacy of drug treatment.

The peptides of the invention exhibit excellent solubility, structure and activity profiles and, therefore, are conducive to manufacturing scale-up and wide-spread clinical applications.

In one embodiment, the polypeptides of the present invention further comprise a protecting group. For instance, the polypeptides can be modified so that the R-groups on the constituent amino acids and/or the terminal amino acids are blocked, i.e., protected, by a protecting group. It has been found that blockage, particularly of the amino and/or carboxy termini, can greatly improve oral delivery and significantly increases serum half-life. Thus, in one embodiment, the polypeptides of the present invention further comprise a protecting group coupled to the amino or carboxy terminus. In one embodiment, the polypeptides further comprise a first protecting group coupled to the amino terminus and a second protecting group coupled to the carboxyl terminus.

Suitable protecting groups include, but are not limited to, acetyl (Ac), amide, 3 to 20 carbon alkyl groups, Fmoc, t-butoxycarbonyl (Tboc), 9-fluoreneacetyl group, 1-fluorenecarboxylic group, 9-fluorenecarboxylic group, 9-fluorenone-1-carboxylic group, benzyloxycarbonyl, xanthyl (Xan), trityl (Trt), 4-methyltrityl (Mtt), 4-methoxytrityl (Mmt), 4-methoxy-2,3,6-trimethyl-benzenesulphonyl (Mtr), mesitylene-2-sulphonyl (Mts), 4,4-dimethoxybenzhydryl (Mbh), tosyl (Tos), 2,2,5,7,8-pentamethyl chroman-6-sulphonyl (Pmc), 4-methylbenzyl (MeBzl), 4-methoxybenzyl (MeOBzl), benzyloxy (BzlO), benzyl (Bzl), benzoyl (Bz), 3-nitro-2-pyridinesulphenyl (Npys), 1-(4,4-dimethyl-2,6-dioxocyclohexylidene)ethyl (Dde), 2,6-dichlorobenzyl (2,6-DiCl-Bzl), 2-chlorobenzyloxycarbonyl (2-Cl-Z), 2-bromobenzyloxycarbonyl (2-Br-Z), benzyloxymethyl (Bom), cyclohexyloxy (cHxO), t-butoxymethyl (Bum), t-butoxy (tBuO), t-butyl (tBu), and trifluoroacetyl (TFA).

In a preferred embodiment, the polypeptides comprise a first protecting group coupled to the amino terminus, the first protecting group including, but not limited to, acetyl, propionyl, and a 3 to 20 carbon alkyl. In a preferred embodiment, the first protecting group is an acetyl. In another preferred embodiment, the polypeptides comprise a second protecting group coupled to the carboxyl terminus, the second protecting being a amide.

The polypeptides of the present invention can comprise all “L” amino acids, all “D” amino acids or a mixture of “L” and “D” amino acids. It has been found that polypeptides comprising all D-amino acids stimulate cholesterol efflux with high-capacity and high-affinity like the L-amino acid polypeptides and retain resistance to oxidation.

A further embodiment of the invention provides pharmaceutical compositions comprising at least one oxidation resistant polypeptide described herein and a pharmaceutically acceptable carrier or excipient. In some embodiments, the pharmaceutical compositions comprise an additional therapeutic agent (e.g., a statin such as atorvastatin, lovastatin, pravastatin, simvastatin, fluvastatin, or rosuvastatin; a bile acid binder such as cholestyramine or colestipol; a Nieman-Pick C1-Like 1 sterol transporter channel inhibitor such as Ezetimibe; a platelet clumping inhibitor such as aspirin, ticlopidine, or clopidogrel, niacin/nicotinamide, a PPAR activator, Vitamin E, or combinations thereof, for treating a disease or disorder associated with cholesterol efflux (e.g., cardiovascular disease).

Another aspect of the present invention provides peptidomimetics of the oxidation resistant polypeptides disclosed herein. In one embodiment, the peptidomimetic is a retro-inverso analog. In another embodiment, the peptidomimetic is a retro-enantio analog. In yet another embodiment, the peptidomimetic is a trans-olefin analog. As disclosed herein, the peptidomimetics of the present invention can comprise other back-bone modifications. As with the polypeptides of the present invention, the peptidomimetics of the present invention can further comprise a protecting group and, preferably, a protecting group at both the amino and carboxyl termini.

In a further aspect, the present invention provides a composition comprising an oxidation resistant polypeptide as described herein, e.g., a polypeptide comprising SEQ ID NO: 1, 2, or 3, or a peptidomimetic thereof, complexed with a lipid. In one embodiment, the lipid is a phospholipid. In another embodiment, the phospholipids is 1-palmitoyl-2-oleoyl-sn-glycerol-3-phosphatidylcholine (“POPC”). In yet another embodiment, the composition further comprises a pharmaceutically acceptable carrier.

Yet another aspect of the invention provides methods of mediating cholesterol efflux in a mammalian subject (e.g., a primate such as a human or chimpanzee, or a rodent such as a rat or mouse) by administering at least one oxidation resistant polypeptide or peptidomimetic described herein to the subject. Those of skill in the art will appreciate that a nucleic acid encoding such a polypeptide (or peptidomimetic) can be administered to the subject in lieu of administering the polypeptide (or peptidomimetic). The present invention provides such nucleic acids. Based on their cholesterol efflux activity, the oxidation resistant polypeptides and peptidomimetics of the present invention can be advantageously used to treat, ameliorate or prevent a disease or condition associated with dyslipidemia, hypercholesterolemia and inflammation.

Still another aspect of the present invention provides methods for treating or preventing a symptom of atherosclerosis in a mammal by administering at least one oxidation resistant polypeptide or peptidomimetic described herein to the subject. Again, those of skill in the art will appreciate that a nucleic acid encoding such a polypeptide (or peptidomimetic) can be administered to the subject in lieu of administering the polypeptide (or peptidomimetic). Such nucleic acids are provided by the present invention. In one embodiment of this method, the mammal is a mammal diagnosed as having one or more symptoms of atherosclerosis. In another embodiment, the mammal is diagnosed as at risk for atherosclerosis. Preferably, the mammal is a human, but can also be a non-human animal. In one exemplar embodiment, the polypeptide has an amino acid sequence of SEQ ID NO: 1, 2, or 3.

In another related embodiment, the methods further comprise administering at least one additional therapeutic agent. Examples of such therapeutic agents include, but are not limited to, an antibody, an enzyme inhibitor, an antibacterial agent, an antiviral agent, a steroid, a non-steroidal anti-inflammatory agent, an anti-metabolite, a cytokine, or a soluble cytokine receptor. The enzyme inhibitor may be a protease inhibitor or a cyclooxygenase inhibitor. The additional agent may be added as a part of a pharmaceutical composition, or may be administered concomitantly or within a time period when the physiological effect of the additional agent overlaps with the physiological effect of the polypeptide(s) or peptidomimetic(s) of the present invention. More specifically, an additional agent may be administered concomitantly or one week, several days, 24 hours, 8 hours, or immediately before the administration of the polypeptide(s) or peptidomimetic(s). Alternatively, an additional agent may be administered one week, several days, 24 hours, 8 hours, or immediately after the administration of the polypeptide(s) or peptidomimetic(s).

Yet another aspect of the present invention provides methods for stabilizing a vulnerable plaque, the method comprising administering to a mammal at least one polypeptide or peptidomimetic described herein, e.g., a polypeptide comprising SEQ ID NO:1, 2, or 3. Again, those of skill in the art will appreciate that a nucleic acid encoding such a polypeptide can be administered to the subject in lieu of administering the polypeptide. Such nucleic acids are provided by the present invention. In one embodiment of this method, the mammal is a mammal diagnosed as having one or more vulnerable plaques. In another embodiment, the mammal is diagnosed as at risk for having a vulnerable plaque(s). Preferably, the mammal is a human, but can also be a non-human animal.

The present invention also provides kits for treating or preventing a disease or condition associated with dyslipidemia, hypercholesterolemia or inflammation. In a preferred embodiment, the present invention provides kits for treating or preventing a symptom of atherosclerosis, the kit comprising a container containing a polypeptide or peptidomimetic of the present invention. The kit can further comprise a pharmaceutically acceptable carrier. In addition, the kit can further comprise instructional materials teaching the use of the polypeptide or peptidomimetic for treating or preventing a disease or condition associated with dyslipidemia, hypercholesterolemia or inflammation, such as atherosclerosis. The polypeptides and peptidomimetics provided in the kits of the present invention can comprise all L amino acids, all D amino acids or a mixture of L and D amino acids.

In yet another aspect, the present invention provides use of at least one oxidation resistant polypeptide or peptidomimetic of the present invention in the preparation of a medicament for mediating cholesterol efflux in a mammal. In exemplar embodiments, the polypeptide has an amino acid sequence selected from the group consisting of SEQ ID NOs. 1, 2, or 3 or, alternatively, a peptidomimetic thereof.

In a further aspect, the present invention provides use of at least one oxidation resistant polypeptide or peptidomimetic of the present invention in the preparation of a medicament for treating a symptom of atherosclerosis and/or stabilizing a vulnerable plaque in a mammal. In exemplar embodiments, the polypeptide has an amino acid sequence of SEQ ID NO: 1, 2, or 3, or, alternatively, a peptidomimetic thereof.

Another aspect of the invention provides an isolated nucleic acid encoding a polypeptide of the present invention, an expression vector comprising the nucleic acid, and a host cell comprising the expression vector.

In a further aspect, the invention provides oxidation susceptible and oxidation resistant peptides as described herein that can be used to evaluate disease susceptibility, vulnerable plaque and/or response to therapies.

In addition, a polypeptide or peptidomimetic of the invention can be used for investigating lipoprotein-receptor interactions in animals and animal models, particularly when a polypeptide or peptidomimetic of the present invention is labeled (e.g., radioactive label, fluorescent label, etc.).

A polypeptide or peptidomimetic of the invention can also be used to identify appropriate animal models for elucidation of lipid metabolic pathways. For example, a polypeptide or peptidomimetic can be used to identify animal models and gene and/or drug interactions that have an effect on reverse cholesterol transport.

Other features, objects and advantages of the invention and its preferred embodiments will become apparent from a reading of the detailed description, examples, claims and figures that follow.

### BRIEF DESCRIPTION OF THE DRAWINGS

FIG. 1 provides data showing the sensitivity of ABCA1 efflux peptide ATI-5261 and an analog to acrolein. ATI-5261 was engineered with arginine (R) in place of lysine (K) at positions 5 and 25 (K5, 25/R), creating an oxidation-resistant analog. Panel A. The parent ATI-5261 peptide and K5,25/R analog stimulated cholesterol efflux (4 h) efficiently (no oxidant exposure) from cAMP treatment J774 macrophages. Panel B. Dependence of cholesterol efflux on ABCA1 expression, as judged with and without cAMP (no oxidant exposure). Peptides were used in lipid-free form at 30 mg/ml. Panel C. Peptides were incubated (37° C., 24 h) with acrolein at a molar ratio of 50:1 (acrolein:peptide) in the presence of DTPA; excess aminoguanidine was added to terminate reactions and peptides dialyzed to PBS. Cholesterol efflux (8 h) was assessed using cAMP-treated J774 cells and peptides at 2 mg/ml. Values are % of control efflux with peptides not treated with acrolein.

Panel D. Impact of varying molar ratio of acrolein relative to ATI-5261 on cholesterol efflux from cAMP treated J774 cells.

FIG. 2 provides data showing the % α-helical content of ATI-5261 and the K5, 25→R analog peptide. Peptides in 10 mM phosphate buffer (pH=7.4) were analyzed. ATI-5261 +acrolein sample had significantly lower helical content compared to the ATI-5261 control. The K5,25/R mutant also had a lower α-helical content compared to the ATI-5261 control, however an increase in α-helical content was observed for the acrolein treated K5,25/R mutant. Biosyn. denotes source (commercial vendor) used to synthesize peptides (same for all peptides).

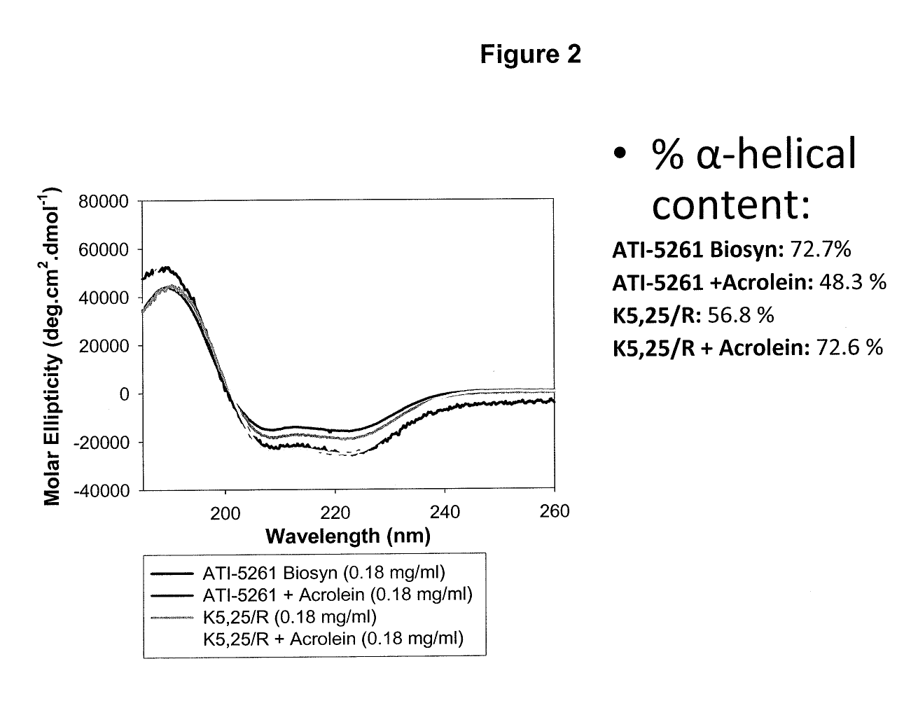

FIG. 3 shows an SDS-PAGE analysis of ATI-5261 and K5,25→R peptides following exposure to acrolein. Peptides ATI-5261 and K5,25 were exposed to 20-fold molar excess of acrolein or buffer alone and incubated 37° C. for 20 h. Lane 1—MW standards (kDa); lane 2, K5,25→R peptide exposed to acrolein; Lane3, ATI-5261 exposed to acrolein; lane 4, K5, 25→R peptide buffer control; Lane 5, ATI-5261 buffer control. Peptide loads were 3 μg/well.

FIG. 4 provides a table that shows the cholesterol efflux activity of Ox-R and Ox-S peptides.

FIG. 5 provides a table that shows the cholesterol efflux activity of Ox-R and Ox-S peptides having a V2→L substitution.

### DETAILED DESCRIPTION OF THE INVENTION

### I. INTRODUCTION

The invention provides oxidation resistant peptide have ABCA1-mediated cholesterol efflux activity. The peptides conform to a motif wherein lysine residues are replaced with arginine residues and are more resistant to oxidation. Accordingly, such peptides can be used in diagnostics, such as for assessment of ABCA pathway function. For example, it has been suggested that ABCA1 protein may be altered in diabetes, i.e. as a result of oxidative damage/glycation. since the peptide itself would be resistant to oxidative damage, it could be used as a way of assessing if ABCA1 pathway is functioning or not.

Further, in vivo reverse cholesterol transport responses, i.e. as determined via a response to Myeloperoxidase(MPO)/oxidation susceptible peptide vs. response to a MPO/oxidation resistant peptide, can be evaluated to identify candidates for treatment and/or to evaluate patient response to treatment.

### II. DEFINITIONS

The term “ABC” or “ATP Binding Cassette” refers to multidomain membrane proteins, responsible for the controlled efflux and influx of allocrites (e.g. cholesterol) across cellular membranes. ABC proteins comprise four domains, with two transmembrane domains (TMDs) responsible for allocrite binding and transport and two nucleotide-binding domains (NBDs) responsible for coupling the energy of ATP hydrolysis to conformational changes in the TMDs. The family members include, e.g., ABCA1 and ABCA7 (see, e.g., Dean et al., J Lipid Res., 42:1007-1017 (2001)). ABCA1 is characterized in Denis et al., J Biol Chem., 279(40):41529-36 (2004). ABCA1 plays a role in cholesterol efflux and is upregulated in cells that are exposed to cholesterol enriching conditions and is the defective molecule in Tangiers Disease (Brooks-Wilson et al., Nat. Gen., 22:336-344 (1999); Bodzioch et al., Nat. Gen., 22:347-351 (1999); Rust et al., Nat. Gen., 22:352-355 (1999)). ABCA1 turns over rapidly and has a half life of about 1 hour in the absence of a suitable stabilizer, such as an apolipoprotein (see, e.g., Wang et al., J. Clin. Invest., 111:99-107 (2003)) ABCA1 sequences are set forth in Genbank Accession Nos.: AJ012376; NM_{—}173076; NM_{—}015657; NM_{—}005502; NP_{—}005493; O95477. The promoter structure and genomic organization of the human ABCA7 gene is described in Broccardo et al., Cytogenet Cell Genet., 92(3-4):264-70 (2001). ABCA7 sequences are set forth in Genbank Accession Nos.: NM_{—}033308; NM 019112; NP_{—}150651; NP_{—}061985; AAK00959. A family of related ATP-binding proteins has been characterized (see, e.g., Higgins et al., J Bioenerg Biomembr., 22(4):571-92 (1990); Higgins et al., Bioessay, 8(4):111-6 (1988); Higgins et al., Nature, 323(6087):448-50 (1986); Doolittle et al., Nature, 323(6087):451-3 (1986); and Blight and Holland, Mol Microbiol., 4(6):873-80 (1990)). The proteins belonging to this family also contain one or two copies of the ‘A’ consensus sequence (see, e.g., Walker et al., EMBO, 1(8):945-51 (1982)) or the ‘P-loop’ (see, e.g., Saraste et al., Trends Biochem Sci., 15(11):430-4 6155 (1990)). ABCA family members are reviewed in Broccardo et al., Biochimica et Biophysica Acta, 1461:395-404 (1999).

The term “oxidation-resistant” as used in the context of this invention refers to a peptide that retains minimum activity (above background) when a non-resistant form is used for comparison, and that preferentially has substantial activity (50-150%) of the activity in the absence of oxidant. A “non-resistant form” of a peptide refers to an “oxidation-sensitive” (also referred to as “oxidation-susceptible”) peptide that comprises one or more lysine residues present as a positively charged residue at the lipid/water interface of an amphipathic alpha helix. Such a peptide also typically comprises at least one tryptophan or tyrosine residues. A “counterpart” oxidation-sensitive polypeptide refers to a peptide that is of almost the same composition, e.g., has at least 85%, 90%, 95%, or greater, identity to an oxidation-resistant polypeptide, but which comprises at least one lysine residue, or in some embodiments, all lysine residues, in place of the arginine residues at the lipid/water interface. The oxidation-sensitive counterpart polypeptide also typically comprises at least one tryptophan residue and/or at least one tyrosine residue.

The term “amphipathic alpha helix” or “amphipathic a helix” refers to a polypeptide sequence that can adopt a secondary structure that is helical with one surface, i.e., face, being polar and comprised primarily of hydrophilic amino acids (e.g., Asp, Glu, Lys, Arg, His, Gly, Ser, Thr, Cys, Tyr, Asn and Gln), and the other surface being a nonpolar face that comprises primarily hydrophobic amino acids (e.g., Leu, Ala, Val, Ile, Pro, Phe, Trp and Met) (see, e.g., Kaiser and Kezdy, Ann. Rev. Biophys. Biophys. Chem., 16:561 (1987), and Science, 223:249 (1984)).

The polar face of an amphipathic a helix can, in some instances, display an “alignment of negatively charged amino acids” or “an alignment of acidic amino acids,” i.e., a series of negatively charged or acidic amino acids (e.g., Asp and/or Glu) positioned approximately evenly (e.g., at about every one, two or three helical turns) within the polypeptide secondary structure. Amphipathic α helices play a role in both intra- and inter-molecular protein-protein interactions, and proteins and lipoproteins (e.g., including apolipoproteins) comprising amphipathic α helices have been postulated to play a role in lipid (e.g., HDL) transport and metabolism (see, e.g., Anantharamaiah et al., Adv. Exp. Med. Biol., 285:131-40 (1991)). The structure and function of amphipathic α helices has been reviewed in, e.g., Segrest et al., Proteins, 8(2):103-17 (1990). In silico methods of identifying amphipathic α helices have been described by, e.g., Jones et al., J. Lipid Res., 33(2):141-66 (1992). Multiple proteins comprising amphipathic α helices have been identified including, e.g., apolipoproteins and serum amyloid proteins.

A structure that is “substantially similar to a three-dimensional conformation” of a polypeptide of the invention refers to structure that comprises a core sequence, e.g., of 24 residues in length, that adopts an amphipathic a helix secondary structure that has an amphipathic orientation of amino acids along the axis of the α-helix structure, with one surface, i.e., face, being polar and comprised primarily of hydrophilic residues and the other surface being a nonpolar face that comprises primarily hydrophobic residues. Two separate acidic residue foci are present along the hydrophilic axis. A polypeptide or peptidomimetic that has a structure substantially similar to a three-dimensional conformation of a polypeptide of the invention also has the ability to stimulate ABCA1-mediated cholesterol efflux.

The term “apolipoprotein” or “Apo” or “exchangeable apolipoprotein” refers to any one of several water soluble proteins that combine with a lipid (i.e., solubilize the lipid) to form a lipoprotein and are constituents of chylomicrons, HDL, LDL and VLDL. Apolipoproteins exert their physiological effect on lipid metabolism by binding to and activating specific enzymes or lipid-transfer proteins or cell-surface receptors or ATP binding cassette transporters (e.g., ABC transporters). The interaction between apolipoproteins and ABCA1 produces cholesterol efflux and HDL particle assembly. Apolipoproteins include, e.g., Apo A-I, Apo A-II, Apo A-IV, Apo C-I, Apo C-II, Apo C-III, Apo E, and serum amyloid proteins such as, serum amyloid A.

The term “Apolipoprotein AI” or Apo A-I refers to a polypeptide comprising 243 amino acids forming N- and C-terminal domains (see, e.g., Saito et al., J. Biol. Chem., 278:23227-23232 (2003) and Saito et al., Prog. Lipid Res., 43:350-380 (2004)). The tertiary structure of apoA-I comprises an N-terminal four-helix bundle domain and a C-terminal domain that binds lipid strongly (see, e.g., Saito et al., Prog. Lipid Res., 43:350-380 (2004) and Mishra et al., Biochemistry, 37:10313-10324 (1998)). Residues 44-243 of apoA-I contain the necessary structural determinants for mediating cholesterol efflux via ABCA1 (see, e.g., Chroni et al., J. Biol. Chem., 278:6719-6730 (2003) and Natarajan et al., J. Biol. Chem., 279:24044-24052 (2004)). This region of apoA-I (aa44-243) is comprised of a series of ten amphipathic α-helices of 11- and 22-amino acids separated by proline residues, as defined by exon 4 of the apoA-I gene (see, e.g., Borhani et al., Proc. Natl. Acad. Sci., 94:12291-6 (1997)). The individual α-helical segments of apoA-I are defined, in part, by the relative distribution of positively charged residues and are designated as Class A or Y (see, e.g., Saito et al., J. Biol. Chem., 278:23227-23232 (2003)). Class A helices possess positively charged amino acids at the lipid-water interface, while class Y helices exhibit a positively charged amino acid toward the middle of the polar surface in addition to interfacial cationic residues. The intact apoA-I molecule has been crystallized, along with a truncated form of the protein (A-I Δ1-43) (see, e.g., Ajees et al. PNAS, 103:2126-2131 (2006); Borhani et al., Acta Crystallogr. D. Biol. Crystallogr., 55:1578-1583 (1999) and Segrest et al., J. Biol Chem., 274:31755-31758 (1999)). Apo AI sequences are set forth in, e.g., Genbank Accession Nos.: P02647, J0009; AAB64381; AAB22835; 1613168A; 1403292A; CAA25519; CAA26097; and LPHUA1.

The terms “cholesterol efflux” and “cholesterol efflux activity” refer to efflux of cholesterol from any cell type. For example, macrophage foam-cells in the artery wall release (i.e., export) cholesterol to appropriate acceptors, such as apolipoproteins and/or HDL. A compound that mediates cholesterol efflux enhances the release, i.e., movement, of cholesterol out of the cell and into the extracellular medium or compartment. Cholesterol efflux is often accompanied by or preceded by, i.e., follows, the efflux of phospholipids from cells. The coordinated release of both cholesterol and phospholipids produces HDL in the presence of a suitable lipid acceptor, e.g., apolipoprotein or peptide. Therefore, the processes of cholesterol- and phospholipid-efflux are linked and synonymous with one another. A compound that enhances the release of cholesterol from cells increases the amount of cholesterol and/or phospholipids appearing outside the cell by at least 25%, 50%, 75%, 100% or by at least 2-fold, 4-fold, 8-fold, 10-fold or more compared to the level of cholesterol efflux in the absence of the compound. An oxidation resistant peptide that has increased activity not only has increased activity in comparison to a counterpart oxidation sensitive peptide, but also can have increased activity in the presence of an agent such as acrolein in comparison to the activity of the oxidation-resistant peptide without acrolein.

The term “ABCA stabilization activity” or “ABCA1 stabilization” refers to enhancing and/or extending the half life of an ABCA protein by preventing its degradation. A compound that has ABCA1 stabilization activity will significantly delay the proteins degradation. This will produce an increase in cellular ABCA1 protein levels of at least 25%, 50%, 75%, 100% or at least 2-fold, 4-fold, 8-fold, 10-fold or higher compared to ABCA1 protein detected in the absence of the compound.

“Plaque stabilization,” as used herein, refers to the stabilization of vulnerable plaques from risk of rupture or erosion by removing cholesterol from lipid rich plaques, including but not limited to, removal of cholesterol from foam cell macrophages. Plaques contain thrombogenic substances, i.e., substances that when exposed to plasma are very powerful in aggregating platelets with the risk of local thrombosis and vessel occlusion, such as tissue factor. The rupture of the plaque and exposure of such material is prevented by the fibrous cap separating the plaque from the vessel. Lipid removal confers plaque stability in two main ways. Firstly, anatomically, lipid removal by shrinking the gruel in the artery is conferring plaque stability by decreasing the risk of hemodynamical stress (expansion-contraction associated with heart beats and blood pressure changes). Secondly, as described in the literature, cholesterol accumulation is stimulating the synthesis and secretion of proteases, including matrix-metallo-proteinases (MMPs) having lysis effects on the fibrous cap.

“Reverse Cholesterol Transport (RCT),” as used herein, refers to the process of removing cholesterol from macrophage foam cells and the lipid rich plaque from the arterial wall, with subsequent transfer through plasma to the liver for uptake, processing and excretion as neutral sterols (cholesterol) or acidic sterols (hydroxylated cholesterol/bile) in feces. The efflux of cholesterol from macrophage foam cells is a requirement for RCT benefit in itself even though the cholesterol may be shifted to other less vulnerable adjacent cells. However, the further disposal of such cholesterol by transport in HDL-like particles to the liver for excretion is a favorable aspect of treatment. Such complete RCT provide a general rejuvenation of the arterial tree by actual net removal of the cholesterol content in the arteries. The RCT and plaque stabilizing effects are either conferred directly by the peptides, or the complexes that they naturally form with phospholipids in plasma and cells or, alternatively, apoA-I/HDL as the peptides bind to endogenous HDL particles, thereby changing their properties and making them more efficient to promote RCT.

A disease or disorder associated with “dyslipidemia” is any disease or disorder in which lipid metabolism is disregulated, due to alterations in tissue (i.e., blood) lipids and lipoprotein concentrations and/or aberrant mediation of cholesterol efflux or aberrant ABCA stabilization. Such diseases include, for example, heart disease, atherosclerotic lesions, stroke, Alzheimer's, and storage disorders.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified polypeptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. A more detailed description of amino acid as well as conservative amino acid substitutions is provided below in the section entitled “Polypeptides.”

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Amino acid polymers may comprise entirely L-amino acids, entirely D-amino acids, or a mixture of L and D amino acids. The use of the term “peptide or peptidomimetic” in the current application merely emphasizes that peptides comprising naturally occurring amino acids as well as modified amino acids are contemplated.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “identical” or percent “identity,” in the context of two or more polypeptide sequences (or two or more nucleic acids), refer to two or more sequences or subsequences that are the same or have a specified percentage of amino acid residues or nucleotides that are the same e.g., 60% identity, preferably 65%, 70%, 75%, 80%, 85%, 90%, 91%, 92%, 93%, 94%, 95%, 96%, 97%, 98%, 99% or 100% identity over a specified region, e.g., SEQ ID NO:3, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, polypeptide-nucleic acids (PNAs). Unless otherwise indicated, a particular nucleic acid sequence also encompasses “conservatively modified variants” thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, mRNA, oligonucleotide, and polynucleotide.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

A “label” or “detectable label” is a composition detectable by spectroscopic, photochemical, biochemical, immunochemical, or chemical means. For example, useful labels include radioisotopes (e.g., ³H, ³⁵S, ³²P, ⁵¹Cr, or ¹²⁵I), fluorescent dyes, electron-dense reagents, enzymes (e.g., alkaline phosphatase, horseradish peroxidase, or others commonly used in an ELISA), biotin, digoxigenin, or haptens and proteins for which antisera or monoclonal antibodies are available (e.g., the polypeptide encoded by SEQ ID NOS: 1, 2, or 3 can be made detectable, e.g., by incorporating a radiolabel into the polypeptide, and used to detect antibodies specifically reactive with the polypeptide).

As used herein, “ameliorates” means alleviate, lessen, or decrease the extent of a symptom or decrease the number of occurrences of episodes of a disease manifestation.

The term “preventing” is art-recognized, and when used in relation to a condition, such as recurrence or onset of a disease such as hypercholesterolemia or atherosclerosis, is well understood in the art, and includes administration of a composition which reduces the frequency of, or delays the onset of, symptoms of a medical condition in a subject relative to a subject which does not receive the composition.

As used herein, “treating” means either slowing, stopping or reversing the progression of the disorder or disease. In a preferred embodiment, “treating” means reversing the progression to the point of eliminating the disorder or disease.

As used herein, “inhibits” means that the amount is reduced as compared with the amount that would occur in a control sample. In a preferred embodiment, inhibits means that the amount is reduced by more than 50%, even more preferably by more than 75% or even 100%.

A “subject,” “patient” or “mammal” to be treated by the methods disclosed herein can mean either a human or non-human animal.

### III. POLYPEPTIDES

The present invention provides a family of non-naturally occurring polypeptides that use the potent Reverse Cholesterol Transport (RCT) pathway to mediate cholesterol efflux and exhibit resistance to oxidation.

The peptides of the invention are based on the surprising discovery of a core amino acid sequence that has an effect on cholesterol efflux, and amino acids within that core that can be substituted to increase resistance to oxidation. In some embodiments, the polypeptides of the present invention are non-naturally occurring polypeptide variants of that core peptide (i.e., the polypeptide of SEQ ID NO:4 (EVRSKLEEWFAAFREFAEEFLARLKS), which is also referred to herein as “ATI-5261” or “5261”) that stimulated ABCA1-dependent cholesterol efflux with a molar potency similar to that of apolipoproteins (e.g., Apo A-I, Apo E, etc.). Interestingly, the polypeptide family members of the present invention are small in size, corresponding to a single helical segment that captures the full biological activity and potency of intact apolipoproteins and the long stretches of multiple α-helical segments found in nature that are required to exert cholesterol efflux activity via ABCA1.

Regarding amphipathic α-helix peptides, hydrophobic amino acids are concentrated on one side of the helix, usually with polar or hydrophilic amino acids on the other. This arrangement is common in alpha helices of apolipoproteins and globular proteins, where one face of the helix is oriented toward the hydrophobic core and one face is oriented toward the water-exposed surface. Different amino-acid sequences have different propensities for forming α-helical structure. Methionine, alanine, leucine, glutamate, and lysine all have especially high helix-forming propensities, whereas proline, glycine, tyrosine, and serine have relatively poor helix-forming propensities. Proline tends to break or kink helices because it cannot donate an amide hydrogen bond (having no amide hydrogen), and because its side chain interferes sterically, thus glycine and serine are often selected as residues in an oxidation-resistant polypeptide.

It will be readily understood by those of skill in the art that the foregoing polypeptides are not fully inclusive of the family of polypeptides of the present invention. In fact, using the teachings provided herein, other suitable polypeptides (e.g., conservative variants) can be routinely produced by, for example, conservative or semi-conservative substitutions (e.g., D replaced by E), extensions, deletions and the like. In addition, using the assays provided herein, other suitable polypeptides can be routinely screened for desired biological activities.

In the current invention, oxidation resistant polypeptides having cholesterol efflux activity typically do not comprise any of the following residues: K, W, Y, C, or M.

The invention additionally provides oxidation resistant peptide based on SEQ ID NO:4 that have R residues at positions 5 and 25. In typical embodiments, an L residue is at position 9. Again, such peptides typically do not comprise any of the following residues: K, W, Y, C, or M. With regard to the presence of L at position 9, it is noted that substitution of W for L in ApoA-I results in loss of activity (see, e.g., Peng et al., Arterioscler. Thromb. Vasc. Biol. 28:2063-2070, 2008J).

The term “percent identical” refers to sequence identity between two amino acid sequences (or between two nucleotide sequences, which are also provided by the present invention). Identity can each be determined by comparing a position in each sequence that may be aligned for purposes of comparison. When an equivalent position in the compared sequences is occupied by the same amino acid or base, then the molecules are identical at that position; when the equivalent site is occupied by the same or a similar amino acid residue (e.g., similar in steric and/or electronic nature), then the molecules can be referred to as homologous (similar) at that position. Expression as a percentage of homology, i.e., similarity, or identity refers to a function of the number of similar or identical amino acids at positions shared by the compared sequences. Various alignment algorithms and/or programs can be used, including, for example, FASTA, BLAST and ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

In another embodiment, which can overlap with the embodiments described above, the polypeptide of SEQ ID NO:3 is substituted with conservative (or semi-conservative) amino acid residues, where the selected residues for an oxidation resistant polypeptide are not K, W, Y, or C. The term “conservative amino acid substitutions” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (see, e.g., Schulz, G. E. and R. H. Schirmer, Principles of Protein Structure, Springer-Verlag). According to such analyses, groups of amino acids may be defined where amino acids within a group exchange preferentially with each other and, therefore, resemble each other most in their impact on the overall protein structure (see, e.g., Schulz, G. E. and R. H. Schirmer, Principles of Protein Structure, Springer-Verlag). One example of a set of amino acid groups defined in this manner include: (i) a charged group, consisting of Glu and Asp, Lys, Arg and His; (ii) a positively-charged group, consisting of Lys, Arg and His; (iii) a negatively-charged group, consisting of Glu and Asp; (iv) an aromatic group, consisting of Phe, Tyr and Trp; (v) a nitrogen ring group, consisting of His and Trp; (vi) a large aliphatic nonpolar group, consisting of Val, Leu and Ile; (vii) a slightly-polar group, consisting of Met and Cys; (viii) a small-residue group, consisting of Ser, Thr, Asp, Asn, Gly, Ala, Glu, Gln and Pro; (ix) an aliphatic group consisting of Val, Leu, Ile, Met and Cys; and (x) a small hydroxyl group consisting of Ser and Thr.

In another exemplary embodiment, which again can overlap with the embodiments described above, “a conservative amino acid substitution” can refer to the substitution of an amino acid for another that is similar in molecular weight or similar in hydrophobicity. By “similar molecular weight” and “similar hyrdrophobicity” is meant a value that is within 25%, more preferably 20%, 15%, 10%, or less than 10% of the respective value. Data for amino acid molecular weights and hydrophobicities are set forth in Table 1. A hydrophobicity ranking is set forth in Table 2; a conservative substitution includes exchanging an amino acid that is designated “=” to another (e.g., Tyr=Trp) and exchanging one amino acid for another that is adjacent to it in the ranking order as delineated by the greater and lesser than symbols.

TABLE 1
Parameters for the Unmodified Physiological L-alpha-Amino Acids

| | 3-Letter | | Molecular | |
|---|---|---|---|---|
| Amino Acid | Code | 1-Letter Code | Weight^{†} | Hydrophobicity^{‡} |
| Alanine | Ala | A | 89.09 | 0.616 |
| Cysteine | Cys | C | 121.16 | 0.680 |
| Aspartate | Asp | D | 133.10 | 0.028 |
| Glutamate | Glu | E | 147.13 | 0.043 |
| Phenylalanine | Phe | F | 165.19 | 1.00 |
| Glycine | Gly | G | 75.07 | 0.501 |
| Histidine | His | H | 155.16 | 0.165 |
| Isoleucine | Ile | I | 131.18 | 0.943 |
| Lysine | Lys | K | 146.19 | 0.283 |
| Leucine | Leu | L | 131.18 | 0.943 |
| Methionine | Met | M | 149.21 | 0.738 |
| Asparagine | Asn | N | 132.12 | 0.236 |
| Proline | Pro | P | 115.13 | 0.711 |
| Glutamine | Gln | Q | 146.15 | 0.251 |
| Arginine | Arg | R | 174.20 | 0.000 |
| Serine | Ser | S | 105.09 | 0.359 |
| Threonine | The | T | 119.12 | 0.450 |
| Valine | Val | V | 117.15 | 0.825 |
| Tryptophan | Trp | W | 204.23 | 0.878 |
| Tyrosine | Tyr | Y | 181.19 | 0.880 |

^{†}The molecular weights given are those of the neutral, free amino acids; residue weights can be obtained by subtraction of one equivalent of water (18 g/mol).
^{‡}The hydrophobicities given are the “Scaled” values from computational log(P) determinations by the “Small Fragment Approach” (see, “Development of Hydrophobicity Parameters to Analyze Proteins Which Bear Post-or Cotranslational Modifications” Black, S. D. and Mould, D. R., Anal. Biochem., 193: 72-82 (1991)). The equation used to scale raw log(P) values to the scaled values given is as follows: Scaled Parameters = (Raw Parameters + 2.061)/4.484.

TABLE 2
Trend of Hydrophobicity Parameters for the Physiological
L-alpha-Amino Acids

Phe > Leu = Ile > Tyr = Trp > Val > Met > Pro > Cys > Ala > Gly >
Thr > Ser > Lys > Gln > Asn > His > Glu > Asp > Arg

Another indication that two polypeptides are conservative variants of one another is that the two polypeptides carry out the same function and, in preferred embodiments, the same function at the same or very similar level of activity. Thus, in one embodiment, a conservative variant of a polypeptide of this invention will comprise an activity of at least 80%, 85%, 86%, 87%, 88%, 89%, 90%, 91%, 92%, 93%, 94%, 95%, 96%, 97%, 98%, 99%, or 100% of that found in a polypeptide of SEQ ID NO:3. Similarly, in other embodiments, a conservative variant of a polypeptide of this invention will comprise an activity of at least 80%, 85%, 86%, 87%, 88%, 89%, 90%, 91%, 92%, 93%, 94%, 95%, 96%, 97%, 98%, 99%, or 100% of that found in a polypeptide of SEQ ID NO:3. Again, in some embodiments, the polypeptides of this invention will possess more than one activity. For example, a polypeptide of the invention can comprise cholesterol efflux mediating activity, ABCA stabilization activity, anti-inflammatory activity, any combination of these activities or, ideally, all of these activities. Conservative variants can have one or more of the same activities and, ideally, all of the same activities. The screening assays described herein can be readily used by those of skill in the art to determine whether two or more polypeptides possess similar activities. In addition, those of skill in the art will know of other screening assays that can be used to determine whether two or more polypeptides possess similar biological properties or activities. In some embodiments, an oxidation-resistant polypeptide of the invention has increased activity relative to an oxidation-sensitive counterpart polypeptide.

While in preferred embodiments, the polypeptides of this invention utilize naturally-occurring amino acids or D forms of naturally occurring amino acids, substitutions with non-naturally occurring amino acids (e.g., methionine sulfoxide, methionine methylsulfonium, norleucine, episilon-aminocaproic acid, 4-aminobutanoic acid, tetrahydroisoquinoline-3-carboxylic acid, 8-aminocaprylic acid, 4-aminobutyric acid, Lys(N(epsilon)-trifluoroacetyl), α-aminoisobutyric acid, and the like) can be used in the polypeptides of the present invention. As with the other amino acid substitutions, non-naturally occurring amino acids are typically substituted so that, upon substitution, they retain the spatial and ionic or non-ionic character of the residue that they substitute.

In addition to the foregoing, the present invention provides truncated forms of the polypeptides of SEQ ID NOS: 1-3. In one such embodiment, the amino acids at positions 25 (i.e., R) and 26 (i.e., S) of SEQ ID NOS. 2 and 3 are not present. The resulting polypeptides, are 24 amino acids in length and have properties similar to the polypeptides of SEQ ID NO:2 and 3.

One of skill understands that amino acid residues may be added to either the C-terminus and/or N-terminus of the polypeptides of the present invention without effecting the activity of such polypeptides. Thus, a polypeptide of the invention that comprises a helical sequence as described herein (e.g., SEQ ID NO:1), includes embodiments that are over 26 amino acids in length, e.g., peptide that are 28, 30, 32, 35, or 40 amino acids in length. One of skill also understands that polypeptides of the invention may also be linked, e.g., via a proline or other linker residues, to another amphipathic a helical peptide that can stimulate cholesterol efflux to form longer polypeptides, e.g., of 50, 60, 70, 80, 90, or 100 amino acids in length. Accordingly, a sequence of any of SEQ ID NOs.1, 2, or 3 can have amino acid additions or can be joined. For example, one molecule of a polypeptide of the invention, e.g., SEQ ID NO: 2 or 3, may be joined to another molecule of the polypeptide through a proline residue to provide a polypeptide that is 53 amino acids in length.

In view of the foregoing, the present invention provides an isolated polypeptide comprising SEQ ID NO:1, 2, or 3, which are resistant to oxidation and have cholesterol efflux activity. In some embodiments, the peptides have increased cholesterol efflux activity relative to an oxidation susceptible counterpart polypeptide, such as SEQ ID NO:4.

In a particularly preferred embodiment, the polypeptides of the present invention comprise one or more D-amino acids as described herein. In certain embodiments, every amino acid (e.g., every enantiomeric amino acid) is a D-amino acid. It has been found that polypeptides comprising all D-amino acids stimulate cholesterol efflux with high-capacity and high-affinity like the L-amino acid polypeptides. D-amino acids are readily incorporated at one or more positions in the polypeptide simply by using a D-form derivatized amino acid residue in the chemical synthesis. D-form residues for solid phase polypeptide synthesis are commercially available from a number of suppliers (see, e.g., Advanced Chem Tech, Louisville, Ky.; Nova Biochem, San Diego, Calif.; Sigma, St Louis, Mo.; Bachem California Inc., Torrance, Calif., etc.). The D-form amino acids can be incorporated at any position in the polypeptide as desired. Thus, for example, in one embodiment, the polypeptide can comprise a single D-amino acid, while in other embodiments, the polypeptide comprises at least two, generally at least three, more generally at least four, most generally at least five, preferably at least six, more preferably at least seven and most preferably at least eight D amino acids. In one embodiment, essentially every other (enantiomeric) amino acid is a D-form amino acid. In certain embodiments, at least 80%, preferably at least 90%, more preferably at least 95% of the enantiomeric amino acids are D-form amino acids. In one particularly preferred embodiment, essentially every enantiomeric amino acid is a D-form amino acid.

In yet another embodiment, peptidomimetics of the polypeptides of the present invention are provided. A “peptidomimetic” includes any modified form of an amino acid chain, including, but not limited to, phosphorylation, capping, fatty acid modifications and including unnatural backbone and/or side chain structures. It will be readily apparent to those of skill in the art that a peptidomimetic comprises the structural continuum between an amino acid chain and a non-peptide small molecule. Peptidomimetics generally retain a recognizable polypeptide-like polymer unit structure. Thus, a peptidomimetic typically retains the function of binding to any target molecule that a natural polypeptide binds to. Examples of suitable peptidomimetics are disclosed in U.S. Patent Application Publication No. 2006/0069030, the teachings of which are incorporated by reference for all purposes. Other peptidomimetics and methods of making same will be known to those of skill in the art.

In preferred embodiments, the peptidomimetics of the present invention fall into one of two categories: (i) surrogates; and (ii) analogs. Numerous surrogates have been developed for the amide bond of polypeptides. Frequently exploited surrogates for the amide bond include, but are not limited to, the following groups: (i) trans-olefins, (ii) fluoroalkene, (iii) methyleneamino, (iv) phosphonamides, and (v) sulfonamides. Examples of such surrogates are disclosed in U.S. Patent Application Publication No. 2006/0069030. Additionally, peptidomimetics based on more substantial modifications of the backbone of a polypeptide can be used. Peptidomimetics that fall in this category include (i) retro-inverso analogs, and (ii) N-alkyl glycine analogs (so-called peptoids). Again, examples of such analogs are disclosed in U.S. Patent Application Publication No. 2006/0069030.

In one embodiment of the present invention, the peptide or peptidomimetic is a retro-inverso analog. Retro-inverso analogs can be made according to the methods known in the art, in a manner similar to synthesizing L-amino acid based polypeptides. More specifically, examples of methods suitable for preparing such retro-inverso analogs are described in U.S. Pat. No. 4,522,752, which issued to Sisto et al. The final product, or inteiniediates thereof, can be purified by HPLC or any other suitable chromatographic method known to those of skill in the art.

In another embodiment, the peptide or peptidomimetic is a retro-enantio analog. Retro-enantio analogs can be synthesized from commercially available D-amino acids (or analogs thereof) using standard solid- or solution-phase polypeptide-synthesis techniques.

In still another embodiment, the peptidomimetic is a trans-olefin analog or derivative. Such trans-olefin analogs of a polypeptide can be readily synthesized according to the method of Shue et al., Tetrahedron Lett., 28:3225 (1987). In addition, other methods known in the art can also be used. It will be appreciated that variations in the procedure of Sjue et al., or other procedures available, may be necessary depending on the nature of the reagents used in synthesizing the trans-olefin derivative.

It is also possible to couple the pseudodipeptides synthesized by the above method to other pseudodipeptides, to make pseudopeptides with several olefinic functionalities in place of amide functionalities. For example, pseudodipeptides corresponding to certain di-peptide sequences can be made and then coupled together by standard techniques to yield an analog of the polypeptide that has alternating olefinic bonds between residues.

Still another class of peptidomimetic derivatives includes phosphonate derivatives. The synthesis of such phosphonate derivatives can be adapted from known synthesis schemes (see, for example, Loots et al. in “Peptides: Chemistry and Biology,” (Escom Science Publishers, Leiden, p. 118, 1988); Petrillo et al. in “Peptides: Structure and Function (Proceedings of the 9th American Peptide Symposium),” (Pierce Chemical Co. Rockland, Ill., 1985).

In other embodiments, the modification can be the introduction of carbohydrate or lipid moieties. Such modifications can change the solubility of the polypeptides in various mediums so that they can advantageously be prepared as a suitable pharmaceutical composition. Modifying lipid groups include, but are not limited to, farnesyl groups and myristoyl groups. Modifying carbohydrate groups include, but are not limited to, single sugars or oligosaccharides of any naturally occurring and/or synthetic sugar and sugar alcohols including, for example, glucose, galactose, rhamnose, mannose, arabinose, and other sugars, and their respective alcohols.

In certain embodiments, the peptidomimetics of the invention may further comprise modifications analogous to post-translational modifications. Such modifications include, but are not limited to, acetylation, carboxylation, glycosylation, phosphorylation, lipidation, and acylation. As a result, the modified peptidomimetics may contain non-amino acid elements, such as polyethylene glycols, lipids, poly- or mono-saccharide, and phosphates. Effects of such non-amino acid elements on the functionality of a peptidomimetic can be tested using the assay methods disclosed herein.

Thus, in a preferred embodiment, the peptidomimetics of the present invention have a three-dimensional conformation that is substantially similar to a polypeptide of SEQ ID NO:1, 2, or 3. In particular embodiments, the peptidomimetics include at least one backbone linkage that is not an amide linkage in the amino to carboxy direction, such as a retro-inverso polypeptide relative to a naturally-occurring polypeptide, or at least one backbone linkage that is not an amide linkage.

The polypeptides as well as the peptidomimetics of the present invention, including, for example, the retro-inverso peptidomimetics, can be modified so that the R-groups on the constituent amino acids and/or the terminal amino acids are blocked, i.e., protected, by a protecting group. It has been found that blockage, particularly of the amino and/or carboxy termini, greatly improves oral delivery and significantly increases serum half-life. As used herein, “protecting group” refers to a temporary substituent that protects a potentially reactive functional group from undesired chemical transformations. Examples of such protecting groups generally include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

A wide number of protecting groups are suitable for this purpose. Such groups include, but are not limited to, acetyl, CH₃—(CH₂)ₙ—CO—, amide, Fmoc, t-butoxycarbonyl (t-BOC), 9-fluoreneacetyl group, 1-fluorenecarboxylic group, 9-fluorenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimethyl-2,6-dioxocyclohexylidene)ethyl (Dde), 2,6-dichlorobenzyl (2,6-DiCl-Bzl), 2-chlorobenzyloxycarbonyl (2-Cl-Z), 2-bromobenzyloxycarbonyl (2-Br-Z), Benzyloxymethyl (Bom), cyclohexyloxy (cHxO), t-butoxymethyl (Bum), t-butoxy (tBuO), t-Butyl (tBu), and Trifluoroacetyl (TFA). The variable “n” is an integer from 0 to 12, typically 0 to 6 such as 0 to 4. Other suitable protecting groups are disclosed in U.S. Pat. No. 6,933,279, the teachings of which are incorporated by reference.

In one embodiment, preferred protecting groups include, but are not limited to, acetyl, amide, and alkyl groups with acetyl and alkyl groups being particularly preferred for N-terminal protection and amide groups being particularly preferred for carboxyl terminal protection. In one preferred embodiment, an acetyl group is used to protect the amino terminus and an amide group is used to protect the carboxyl terminus. In this embodiment, acetylation can be accomplished during the synthesis when the polypeptide is on the resin using acetic anhydride. Amide protection can be achieved by the selection of a proper resin for the synthesis. For instance, a rink amide resin can be used. After the completion of the synthesis, the semipermanent protecting groups on acidic bifunctional amino acids, such as Asp and Glu, and basic amino acids, such as Lys, as well as the hydroxyl of Tyr, are all simultaneously removed. The polypeptides released from such a resin using acidic treatment comes out with the N-terminal protected as acetyl and the C-terminal protected as NH₂, with the simultaneous removal of all of the other protecting groups.

A. Chemical Synthesis

The polypeptides can be chemically synthesized using methods well known in the art including, e.g., solid phase synthesis (see, e.g., Merrifield, J. Am. Chem. Soc., 85:2149-2154 (1963) and Abelson et al., Methods in Enzymology, Volume 289: Solid-Phase Peptide Synthesis (1st ed. 1997)). Polypeptide synthesis can be performed using manual techniques or by automation. Automated synthesis can be achieved, for example, using Applied Biosystems 431A Peptide Synthesizer (Perkin Elmer). Alternatively, various fragments of the polypeptide can be chemically synthesized separately and then combined using chemical methods to produce the full length polypeptide. The sequence and mass of the polypeptides can be verified by GC mass spectroscopy. Once synthesized, the polypeptides can be modified, for example, by N-terminal acetyl- and C-terminal amide-groups as described above. Synthesized polypeptides can be further isolated by HPLC to a purity of at least about 80%, preferably 90%, and more preferably 95%.

B. Recombinant Expression

The polypeptides described herein can also be expressed recombinantly, especially when the polypeptide does not comprise a “D” amino acid residues. This embodiment relies on routine techniques in the field of recombinant genetics. Generally, the nomenclature and the laboratory procedures in recombinant DNA technology described herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use in this invention include Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 1994)).

Polymerase chain reaction or other in vitro amplification methods may also be useful, for example, to clone nucleic acid sequences that code for the polypeptides to be expressed, to make nucleic acids to use as probes for detecting the presence of encoding mRNA in physiological samples, for nucleic acid sequencing, or for other purposes. Nucleic acids amplified by the PCR reaction can be purified from agarose gels and cloned into an appropriate vector.

Gene expression of a sequence of the invention can also be analyzed by techniques known in the art, e.g., reverse transcription and amplification of mRNA, isolation of total RNA or poly A+ RNA, northern blotting, dot blotting, in situ hybridization, RNase protection, probing DNA microchip arrays, and the like.

To obtain high level expression of a nucleic acid sequence, such as the nucleic acid sequences encoding a polypeptide of this invention, one typically subclones a nucleic acid sequence that encodes a polypeptide sequence of the invention into an expression vector that is subsequently transfected into a suitable host cell. The expression vector typically contains a strong promoter or a promoter/enhancer to direct transcription, a transcription/translation terminator, and for a nucleic acid encoding a protein, a ribosome binding site for translational initiation. The promoter is operably linked to the nucleic acid sequence encoding a polypeptide of the invention or a subsequence thereof. Suitable bacterial promoters are well known in the art and described, e.g., in Sambrook et al. and Ausubel et al. The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to the recombinant polypeptides to provide convenient methods of isolation, e.g., His tags. In some case, enzymatic cleavage sequences (e.g., Met-(His)g-Ile-Glu-GLy-Arg which form the Factor Xa cleavage site) are added to the recombinant polypeptides. Bacterial expression systems for expressing the polypeptides are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Standard transfection methods are used to produce cell lines that express large quantities of polypeptides of the invention, which are then purified using standard techniques (see, e.g., Colley et al., J. Biol. Chem., 264:17619-17622 (1989); Guide to Protein Purification, in Methods in Enzymology, vol. 182 (Deutscher, ed., 1990)). Transformation of cells is performed according to standard techniques (see, e.g., Morrison, J. Bact., 132:349-351 (1977); Clark-Curtiss & Curtiss, Methods in Enzymology, 101:347-362 (Wu et al., eds, 1983). For example, any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing a polypeptide of the invention.

After the expression vector is introduced into the cells, the transfected cells are cultured under conditions favoring expression of a polypeptide of the invention. Polypeptides of the invention are recovered from the culture using standard techniques identified below.

C. Purification of Polypeptides

Polypeptides are purified to substantial purity by standard techniques known in the art, including, for example, extraction and purification from inclusion bodies, size differential filtration, solubility fractionation (i.e., selective precipitation with such substances as ammonium sulfate); column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when polypeptides are being purified. For example, polypeptides having established molecular adhesion properties can be reversible fused to recombinant polypeptides. With the appropriate ligand, the recombinant polypeptides can be selectively adsorbed to a purification column and then freed from the column in a relatively pure form. The fused polypeptide is then removed by enzymatic activity. Finally, the polypeptides may be purified using immunoaffinity columns.

### IV. METHODS OF IDENTIFYING POLYPEPTIDES WITH DESIRED ACTIVITY

The polypeptides or peptidomimetics of the present invention can be readily screened for their resistance to oxidation and the ability to mediate cholesterol efflux and/or stabilize ABCA (e.g., ABCA1) using methods well known to those of skill in the art.

A number of different screening protocols can be utilized to identify polypeptides or peptidomimetics of the present invention that mediate cholesterol efflux and/or stabilize ABCA (e.g., ABCA1). In one embodiment, the screening methods involve screening a plurality of test polypeptides to identify those polypeptides that mediates cholesterol efflux and/or stabilizes ABCA (e.g., ABCA1) in, e.g., mammalian cells, including human cells.

In addition to screening for their ability to mediate cholesterol efflux and/or stabilize ABCA, candidate test polypeptides can also be screened for other activities including, e.g., anti-oxidant activities and anti-inflammatory activities. A number of different screening protocols can be utilized to identify polypeptides or peptidomimetics of the present invention that have anti-oxidant activity and/or anti-inflammatory activity.

It will be readily apparent to those of skill in the art that numerous other screening assays, in addition to those disclosed herein, can be used to screen the polypeptides or peptidomimetics of the present invention for the desired biological activities.

Oxidation resistance can be assessed using methods well-known in the art. For example, an oxidation resistant polypeptide can be tested relative to a counterpart polypeptide peptide that comprises one or more lysine residues at the lipid/water interface of the amphipathic alpha helix for the ability to stimulate cholesterol efflux (no oxidant exposure) from cAMP treatment of macrophages. Peptides can then be tested for cholesterol efflux activity in the presence of an oxidative agent such as acrolein. An oxidation-resistant polypeptide retains activity, e.g., at least 10%, 20%, 25%, 50%, 75%, or even 100% of greater, of the activity of the oxidation susceptible counterpart polypeptide.

A. Screening for Cholesterol Efflux Activity

Suitable cholesterol efflux assays are described in, e.g., Bielicki, J. K and Oda, M. N., Biochemistry, 41:2089-2096 (2002); Jia et al., Biochem. Biophys. Res. Common., 297:206-213 (2002). In some embodiments, a polypeptide known to mediate cholesterol efflux (e.g., helix 9/10 of Apo A-I) is used to screen for additional mediators of cholesterol efflux in a cell based assay. For example, cell lines in which cholesterol efflux can be enhanced using a cAMP analog that up-regulates ABCA1 protein expression (e.g., J774 macrophages) can conveniently be used to assess the ability of a polypeptide of the present invention to mediate cholesterol efflux. The cells are incubated with labeled cholesterol (e.g., [³H]cholesterol) under conditions appropriate for cholesterol uptake by the cells. Thus, cAMP or cAMP analogs (e.g., CPT-cAMP) are incubated with the cells for a suitable time before the initiation of cellular cholesterol efflux, i.e., prior to contacting the cells with a test polypeptide. Measurement of labeled cholesterol appearing in the medium is used to determine the cholesterol efflux mediating activity of the test polypeptide.

B. Screening for ABCA Stabilization Activity

Multiple assays known in the art can be used to measure the ABCA stabilization activity of a polypeptide of the invention. For example, binding assays can be used to test the ability of the test polypeptide to bind to ABCA (e.g., ABCA1). It has been found that polypeptides having ABCA stabilization activity are also likely mediators of cholesterol efflux. As such, in a preferred embodiment, the polypeptides or peptidomimetics of the present invention have the ability to mediate cholesterol efflux and to stabilize ABCA. In one screening embodiment, the binding assays can be competitive assays. Other assays include, for example, direct measurement of ABCA (e.g., ABCA protein or nucleic acids) following contact with the test polypeptide.

1. Binding Assays

Binding assays usually involve contacting ABCA with one or more test polypeptides, and allowing sufficient time for ABCA and the test polypeptides to form a binding complex. Any binding complexes formed can be detected using any of a number of established analytical techniques. Protein binding assays include, but are not limited to, immunohistochemical binding assays, flow cytometry or other assays. In some embodiments, competition assays are used to determine whether a test polypeptide has ABCA stabilization activity. Competition assays are well known in the art. Typically, a competitor compound, i.e., a compound known to bind ABCA, is labeled so that differences in binding to ABCA (e.g., in the presence of increasing amount of a test polypeptide of the invention that may bind to ABCA) can be measured. The particular label or detectable group used in the assay is not a critical aspect of the invention, as long as it does not significantly interfere with the binding of the test compound to ABCA. As described herein, the detectable group (or, alternatively, detectable moiety or label) can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include, but are not limited to, magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

In some embodiments, ABCA expressing and non-expressing cells are used to measure the ABCA (e.g., ABCA1) stabilization activity of a test polypeptide by measuring the relative ABCA binding affinities of the test polypeptide and a competitor compound (e.g., full-length Apo A-I A or Apo A-I 9/10 polypeptide) for ABCA. In some embodiments, the binding affinity of full-length Apo A-I A to ABCA is compared to the binding affinity of a labeled polypeptide of the invention as described in, e.g., Remaley et al., J. Lipid Res., 44:828-836 (2003). Cells expressing ABCA are incubated in the presence and absence of the competitor compound, and then exposed to a range of concentrations of individual labeled test polypeptides (e.g., a radiolabeled polypeptide of the invention). Typically, the concentrations of test polypeptides will range from about 0.1 μm/ml to about 200 μm/ml, about 0.5 μg/ml to about 100 μm/ml, about 1 μm/ml to about 40 μm/ml, or about 5 μg/ml to about 20 μg/ml.

2. Direct Measurement of ABCA

In some embodiments, the stabilization of ABCA is measured by direct measurement of ABCA (e.g., ABCA protein, or nucleic acid) using a cell based assay. Cell based assays can be performed in any cells in which ABCA is expressed (e.g., J774 macrophages), including cells which have been transfected with ABCA (e.g. HeLa cells). Any cell type can be used. For example, J774 macrophages can be used to assess relative ABCA1 protein levels in the presence and absence of polypeptides of the invention. The cells are first contacted with a compound that will induce ABCA (e.g., cAMP or a cAMP analogue such as, 8-bromo-cAMP) to upregulate ABCA (e.g., ABCA1) expression, then exposed to synthetic ABCA1 protein levels in the presence and absence of polypeptides of the invention in the absence of the cAMP stimulus to evaluate whether ABCA1 protein was stabilized or degraded. Relative levels of ABCA1 protein can be assessed using any means known in the art including, e.g., immunoblot analysis of cell membranes (Oram et al., J. Biol. Chem., 278:52379-52385 (2003)) or hybridization of nucleic acid probes to ABCA mRNA.

C. Further Testing

Polypeptides that are initially identified as mediating cholesterol efflux or interacting with ABCA can be further tested to validate their ability to mediate cholesterol efflux and/or stabilize ABCA. The basic format of such methods involves administering a lead compound identified during an initial screen to an animal that serves as a model. The animal models utilized in validation studies generally are mammals of any kind. Specific examples of suitable animals include, but are not limited to, primates (e.g., chimpanzees, monkeys, and the like) and rodents (e.g., mice, rats, guinea pigs, rabbits, and the like). In a preferred embodiment, Apo E−/−mice, Apo A-II−/−mice, or Apo C-III−/−mice are used. Additional animal models are described in, e.g., Marschang et al., Sem. Cell Dev. Biol., 14:25-35 (2003).

Screening assay can be performed in a high throughput format, e.g., of a combinatorial polypeptide library or peptidomimetics, or any other format.

### V. METHODS OF USE

The non-naturally occurring polypeptides of the present invention use the potent Reverse Cholesterol Transport (RCT) pathway to mediate cholesterol efflux. In addition to being potent and selective mediators of ABCA1-dependent cholesterol efflux, the polypeptides of the present invention are also resistant to oxidation. These properties have both diagnostic and therapeutic uses.

### VI. USE AS RESEARCH TOOLS AND IN METHODS OF DIAGNOSIS

The polypeptides and peptidomimetics of the invention are also useful for diagnostic purposes and as research tools. In particular, oxidation-sensitive and oxidation-resistant peptides can be used diagnostically.

In other embodiments, the polypeptides or peptidomimetics of the invention can be used in methods of diagnosing diseases and disorders associated with aberrant cholesterol efflux or with ABCA. For example, the peptides can be used in assays to diagnose reverse cholesterol transport deficiency and to identify individuals predicted to be responders to peptide treatment. Such diagnostic assays include in vitro assays. For example, cholesterol efflux can be evaluated in an assay in which a polypeptide of the invention, e.g., any one of SEQ ID NO:1, 2, or 3 is mixed with plasma from a subject and exposed to cells to indicate whether a subject would respond to treatment (e.g., a large increase in efflux in the presence of the peptide compared with plasma-mediated efflux in the absence of the peptide suggests that the subject would be responsive). Similarly, a polypeptide of the invention, e.g., any one of SEQ ID NO:1, 2, or 3 can be mixed with plasma from a subject to detect changes in HDL subclass distribution and/or to detect changes in anti-oxidative properties of the plasma in the presence of the peptide.

In some embodiments, the polypeptides or peptidomimetics are used for in vivo imaging methods. The polypeptides or peptidomimetics are conjugated to a detectable moiety and administered to a subject (e.g., a mammal such as a human). Detection of the detectable moiety allows imaging of a cell, tissue, or organ of interest, including, e.g., an atherosclerotic lesion or an amyloid plaque.)

The term “imaging” refers to a procedure or modality for generating an image of a detectable moiety in vivo, ex vivo, or in vitro as described herein or known to one of skill in the art. Examples of imaging modalities include, but are not limited to, magnetic resonance, nuclear magnetic resonance, radioscintigraphy, positron emission tomography, computed tomography, near-infrared fluorescence, X-ray, ultra sound, ultraviolet light, or visible light (see, e.g., Dahnhert, Radiology Review Manual (4th ed. 1999); Brant et al., Fundamentals of Diagnostic Radiobiology (2nd ed. 1999); Weissleder et al., Primer of Diagnostic Imaging (2nd ed. 1997); Buddinger et al., Medical Magnetic Resonance A Primer, Society of Magnetic Resonance, Inc. (1988); and Weissleder et al., Nature Biotech., 17:375-378 (1999)).

The phrase “detectable moiety,” as used herein, refers to a moiety or label that can be imaged and/or detected in vivo, ex vivo, or in vitro by a procedure or modality described herein or known to one of skill in the art. As used herein, the detectable moiety can be directly or indirectly linked to a polypeptide or peptidomimetic of the invention. A linker may serve to link the polypeptide or peptidomimetic to one detectable moiety. Alternatively, a linker may link the polypeptide to more than one detectable moiety. Likewise, a detectable moiety may be linked to more than one linker. The use of a plurality of detectable moieties attached to one polypeptide enables the detectability of the detectable moiety to be increased (e.g., by increasing its radiopacity, echogenicity or relaxivity) or, alternatively, it may enable the polypeptide to be detected in more than one imaging modality.

Linking of a detectable moiety to a polypeptide or peptidomimetic of the invention may be achieved by covalent or non-covalent means, usually involving interaction with one or more functional groups located on the detectable moiety, the linker and/or the polypeptide. Examples of chemically reactive functional groups that may be employed for this purpose include, but are not limited to, amino, hydroxyl, sulfhydryl, carboxyl, and carbonyl groups, as well as carbohydrate groups, vicinal dials, thioethers, 2-amino alcohols, 2-amino thiols, guanidinyl, imidazolyl and phenolic groups. In some embodiments, labile linkages, e.g., containing spacer arms that are biodegradable or chemically sensitive or which incorporate enzymatic cleavage sites, are used. The particular linker is not a critical aspect of the invention. Any linker known in the art may be used as long it binds the polypeptide or peptidomimetic and the detectable moiety together for an adequate period, i.e., a period sufficient for the polypeptide the desired target and be detected.

The detectable moieties used in the methods of the present invention can be any moiety capable of detection either directly or indirectly in an imaging procedure described herein or known to one of skill in the art. For example, the following detectable moieties may be used: moieties which emit or may be caused to emit detectable radiation (e.g., by radioactive decay, fluorescence excitation, spin resonance excitation, etc.), moieties which affect local electromagnetic fields (e.g., paramagnetic, superparamagnetic, ferrimagnetic or ferromagnetic species), moieties which absorb or scatter radiation energy (e.g., chromophores, particles (including gas or liquid containing vesicles), heavy elements and compounds thereof, etc.), and moieties which generate a detectable substance (e.g., gas microbubble generators).

A very wide range of materials detectable by imaging modalities is known from the art and the detectable moiety will be selected according to the imaging modality to be used. Thus, for example, for ultrasound imaging, an echogenic material or a material capable of generating an echogenic material will normally be selected; for X-ray imaging, the detectable moiety will generally be or contain a heavy atom (e.g., of atomic weight 38 or above); for MR imaging, the detectable moiety will either be a non zero nuclear spin isotope (such as ¹⁹F) or a material having unpaired electron spins and hence paramagnetic, superparamagnetic, ferrimagnetic or ferromagnetic properties; for light imaging, the detectable moiety will be a light scatterer (e.g., a colored or uncolored particle), a light absorber or a light emitter; for magnetometric imaging, the detectable moiety will have detectable magnetic properties; for electrical impedance imaging, the detectable moiety will affect electrical impedance; and for scintigraphy, SPECT, PET, etc., the detectable moiety will be a radionuclide.

Examples of suitable detectable moieties that are well known from the diagnostic imaging literature include, e.g., magnetic iron oxide particles, gas-containing vesicles, chelated paramagnetic metals (such as Gd, Dy, Mn, Fe, etc.) (see, for example, U.S. Pat. Nos. 5,228,446; 4,647,447; 4,863,715; 4,770,183, and 5,387,080; PCT Publication No. WO 97/25073, WO 96/09840, WO 85/02772, WO 92/17212, WO 97/29783, WO 91/15243, WO 93/05818, WO 96/23524, WO 95/26205 and WO 96/17628; EP-A-554213; and GB 9624918.0; metal radionuclides, paramagnetic metal ions, fluorescent metal ions, heavy metal ions and cluster ions as described in PCT Publication No. WO 91/14460, WO 89/00557, WO 92/17215, WO 96/40287 and WO 96/22914; and U.S. Pat. Nos. 4,647,447, 5,367,080 and 5,364,613; non-metal atomic moieties such as, e.g., ¹²³I, ¹³¹I, and ¹⁸F, and heavy atoms such as I; organic chromophoric or fluorophoric moieties as described in Matsuoka, Topics in Applied Chemistry: Infrared absorbing dyes (1990); Waring et al., Topics in Applied Chemistry: The Chemistry and Application of Dyes (1990); “Handbook of Fluorescent Probes and Research Chemicals” Haugland, Molecular Probes Inc, 1996, DE-A-4445065, DE-A-4326466, JP-A-3/228046, Narayanan et al., J. Org. Chem., 60:2391-2395 (1995), Lipowska et al., Heterocyclic Comm., 1:427-430 (1995), Fabian et al., Chem. Rev., 92:1197 (1992); PCT Publication No. W096/23525: Strekowska et al.,. J. Org. Chem., 57:4578-4580 (1992); and PCT Publication No. WO 96/17628; visible dyes as described in, Waring and Hallas, The Chemistry and Application of Dyes, Topics in Applied Chemistry (1990); Haugland, Handbook of Fluorescent Probes and Research Chemicals (6th ed. 1996).

Examples of imaging modalities suitable for detecting the detectable moiety linked to the ligand include, but are not limited to, magnetic resonance, nuclear magnetic resonance, radioscintigraphy, positron emission tomography, computed tomography, near-infrared fluorescence, X-ray, ultra sound, ultraviolet light, or visible light, wherein the image of the detectable moiety is indicative of the activity of a specific extracellular protease (see, for example, Dahnhert, Radiology Review Manual (4th ed. 1999); Brant et al., Fundamentals of Diagnostic Radiobiology, (2nd ed. 1999); Weissleder et al., Primer of Diagnostic Imaging, (2nd ed. 1997); Buddinger et al., Medical Magnetic Resonance A Primer, Society of Magnetic Resonance, Inc.(1988); and Weissleder et al., Nature Biotech., 17:375-378 (1999)).

In certain circumstances, it may be desirable that the linker biodegrade after administration. By selecting an appropriately biodegradable linker, it is possible to modify the biodistribution and bioelimination patterns for the polypeptide and/or detectable moiety. Where polypeptide and/or detectable moiety are biologically active or are capable of exerting undesired effects if retained after the imaging procedure is over, it may be desirable to design biodegradability into the linker that ensures appropriate bioelimination or metabolic breakdown of the polypeptide and/or detectable moieties. Thus, a linker may contain a biodegradable function that on breakdown yields breakdown products with modified biodistribution patterns that result from the release of the detectable moiety from the polypeptide or from fragmentation of a macromolecular structure. By way of example, for linkers that carry chelated metal ion moieties, it is possible to have the linker incorporate a biodegradable function that on breakdown releases an excretable chelate compound containing the detectable moiety. Accordingly, biodegradable functions may, if desired, be incorporated within the linker structure, preferably at sites which are (a) branching sites, (b) at or near attachment sites for ligands or detectable moieties, or (c) such that biodegradation yields physiologically tolerable or rapidly excretable fragments.

In other embodiments, the polypeptide of the invention may be used as research tools. For example, the polypeptides or peptidomimetics of the invention can be used for investigating lipoprotein-receptor interactions in animals and animal models, particularly when a polypeptide or peptidomimetic thereof is labeled with a detectable moiety, e.g., a radioactive label, a fluorescent label, etc. In addition, the polypeptides of the invention can also be used to identify appropriate animal models for elucidation of lipid metabolic pathways. For example, the polypeptides can be used to identify animal models where lipid peroxidation contributes to the progression of atherosclerosis. Moreover, the polypeptides of the invention can be used to evaluate the anti-atherosclerotic potential of other compounds (including, e.g., polypeptide variants and other peptidomimetics).

A. Treating or Preventing A Symptom(s) of Atherosclerosis

In view of their biological activities and, in particular, their ability to mediate cholesterol efflux, the polypeptides of the present invention (or peptidomimetics thereof) can be used to treat elevated cholesterol levels in a mammal, or to treat prophylactically a mammal at risk of developing elevated cholesterol levels. In addition, the polypeptides or peptidomimetics can also be used for improving the lipid parameters in a mammal. An improvement in “lipid parameters” includes, for example, one or more of a decrease in the propensity of lipoproteins to adhere to a blood vessel, a decrease in the amount of atherosclerotic plaque (even though plasma LDL and/or HDL concentrations may not significantly changed), a reduction in the oxidative potential of an HDL or LDL particle, a regression in atherosclerosis (e.g., as measured by carotid angiography or ultrasound) and a reduction in cardiac events. Thus, the polypeptides or peptidomimetics of the present invention can be used to treat or prevent (i.e., prophylactically treat) diseases and conditions associated with dyslipidemia, hypercholesterolemia and inflammation, or diseases and conditions that are treatable by altering lipid parameters, such as those diseases and conditions disclosed herein.

In addition to the diseases and conditions specifically disclosed herein, those of skill in the art will know of other diseases and conditions associated with dyslipidemia, hypercholesterolemia and inflammation that can be treated or prevented using the polypeptides or peptidomimetics of the present invention.

B. Treating or Preventing A Symptom(s) of Atherosclerosis

In one embodiment, the present invention provides methods for treating, ameliorating and/or preventing one or more symptoms of atherosclerosis. The methods preferably involve administering to an organism, preferably a mammal and, more preferably, a human, one or more of the polypeptides of this invention (or peptidomimetics of such polypeptides). The polypeptide(s) can be administered, as described herein, according to any of a number of standard methods including, but not limited to, injection, suppository, nasal spray, time-release implant, transdermal patch, orally and the like. In one particularly preferred embodiment, the polypeptide(s) is administered orally (e.g., as a syrup, capsule, tablet, etc.).

The methods of the present invention are not limited to treating humans or non-human animals having one or more symptom(s) of atherosclerosis (e.g., hypertension, narrowing of vessels, plaque formation and rupture, heart attack, angina, or stroke, high levels of plasma cholesterol, high levels of low density lipoprotein, high levels of very low density lipoprotein, or inflammatory proteins, etc.), but are also very useful in a prophylactic context. Thus, the polypeptides of this invention (or peptidomimetics thereof) can be administered to an organism, such as a human or non-human animal, to prevent the onset, i.e., development, of one or more symptoms of atherosclerosis. Suitable candidate subjects for prophylactic treatment include, for example, those subjects having one or more risk factors for atherosclerosis (e.g., family history, genetic markers that correlate with atherosclerosis, hypertension, obesity, high alcohol consumption, smoking, high blood cholesterol, high blood triglycerides, elevated blood LDL, VLDL, IDL, or low HDL, diabetes, or a family history of diabetes, high blood lipids, heart attack, angina or stroke, etc.).

Treatment can complement or obviate the need for vascular surgery making anti-atherosclerosis treatment systemic and sustainable. Thus, the peptide can be given before intervention to optimize circulation before surgery, during surgery for regional administration in the vasculature or its vicinity, or post-surgery to lessen inflammation and atherosclerosis caused by mechanical trauma by surgical intervention.

C. Treating or Preventing A Symptom(s) of Atherosclerosis Associated with an Acute Inflammatory Response

The atherosclerosis-inhibiting polypeptides of this invention are also useful in a number of other contexts. In particular, it has been found that cardiovascular complications (e.g., atherosclerosis, stroke, etc.) frequently accompany or follow the onset of an acute phase inflammatory response. Such an acute phase inflammatory response is often associated with a recurrent inflammatory disease (e.g., leprosy, tuberculosis, systemic lupus erythematosus, rheumatoid arthritis, etc.), a viral infection (e.g., influenza, HIV, etc.), a bacterial infection, a fungal infection, an organ transplant, a wound or other trauma, an implanted prosthesis, a biofilm, and the like.

The polypeptides described herein can be used to reduce or prevent the formation of oxidized phospholipids during or following an acute phase inflammatory response, thereby mitigating or eliminating cardiovascular complications associated with such a condition.

Thus, in certain embodiments, this invention contemplates administering one or more of the polypeptides of this invention to a subject at risk for, or incurring, an acute phase inflammatory response and/or at risk for or incurring a symptom of atherosclerosis.

The peptides of the invention effects lipids and thereby can be useful for the treatment of disease states in which lipids and lipid metabolism play a role. Thus, for example, a person having or at risk for coronary disease can prophylactically be administered a polypeptide of this invention during flu season. A human (or other animal) subject to a recurrent inflammatory condition, e.g., rheumatoid arthritis, various autoimmune diseases, etc., can be treated with a polypeptide of this invention to mitigate or prevent the development of atherosclerosis or stroke. Similarly, a human (or other animal) subject to trauma, e.g., acute injury, tissue transplant, etc., can be treated with a polypeptide of this invention to mitigate or prevent the development of atherosclerosis or stroke.

In certain instances, such methods will entail a diagnosis of the occurrence or risk of an acute inflammatory response. The acute inflammatory response typically involves alterations in metabolism and gene regulation in the liver. It is a dynamic homeostatic process that involves all of the major systems of the body, in addition to the immune, cardiovascular and central nervous system. Normally, the acute phase response lasts only a few days; however, in cases of chronic or recurring inflammation, an aberrant continuation of some aspects of the acute phase response may contribute to the underlying tissue damage that accompanies the disease, and may also lead to further complications, for example, cardiovascular diseases or protein deposition diseases such as amyloidosis.

An important aspect of the acute phase response is the radically altered biosynthetic profile of the liver. Under normal circumstances, the liver synthesizes a characteristic range of plasma proteins at steady state concentrations. Many of these proteins have important functions and higher plasma levels of these acute phase reactants (APRs) or acute phase proteins (APPs) are required during the acute phase response following an inflammatory stimulus. Although most APRs are synthesized by hepatocytes, some are produced by other cell types, including monocytes, endothelial cells, fibroblasts and adipocytes. Most APRs are induced between 50% and several-fold over normal levels. In contrast, the major APRs can increase to 1000-fold over normal levels. This group includes serum amyloid A (SAA) and either C-reactive protein (CRP) in humans or its homologue in mice, serum amyloid P component (SAP). So-called negative APRs are decreased in plasma concentration during the acute phase response to allow an increase in the capacity of the liver to synthesize the induced APRs.

In certain embodiments, the acute phase inflammatory response, or risk therefore is evaluated by measuring one or more APPs. Measuring such markers is well known to those of skill in the art, and commercial companies exist that provide such measurement (e.g., AGP measured by Cardiotech Services, Louisville, Ky.). Once it has been determined that a person is experiencing an acute phase inflammatory response or is at risk of experiencing an acute phase inflammatory response, the polypeptides of the present invention can be administered to reduce or prevent the formation of oxidized phospholipids during or following the acute phase inflammatory response, thereby mitigating or eliminating cardiovascular complications associated with such a condition.

D. Treating or Preventing a Symptom(s) or Condition Associated with Coronary Calcification and Osteoporosis

It has also been found that oxidized lipids can be a cause of coronary calcification and osteoporosis. It is also thought that oxidized lipids can be involved in the pathogenesis of calcific aortic stenosis.

Thus, in another embodiment, the polypeptides of the present invention are used to treat, inhibit or prevent a symptom of a disease such as polymyalgia rheumatica, polyarteritis nodosa, scleroderma, idiopathic pulmonary fibrosis, chronic obstructive pulmonary disease, Alzheimers Disease, AIDS, coronary calcification, calcific aortic stenosis, osteoporosis and the like. In such methods, the polypeptides or peptidomimetics of the present invention can be administered to a human or non-human animal to reduce or prevent the formation of oxidized phospholipids, thereby inhibiting or preventing a symptom of a disease such as polymyalgia rheumatica, polyarteritis nodosa, scleroderma, idiopathic pulmonary fibrosis, chronic obstructive pulmonary disease, Alzheimers Disease, AIDS, coronary calcification, calcific aortic stenosis, osteoporosis and the like.

Typically, all of the above methods involve the administration of a single polypeptide of this invention or, alternatively, the administration of two or more different polypeptides of this invention. Such polypeptides can be administered alone or in combination with other therapeutic agents, such as those disclosed herein. The polypeptides can be provided as monomers or in dimeric, oligomeric or polymeric forms. In certain embodiments, the multimeric forms may comprise associated monomers (e.g., ionically or hydrophobically linked); whereas, in other embodiments, other multimeric forms comprise covalently linked monomers (directly linked or through a linker).

In addition, although all of the foregoing methods are described herein with respect to humans, it will be readily apparent to those of skill that such methods are also useful for other animals, i.e., for veterinary use. Thus, preferred organisms include, but are not limited to, humans, non-human primates, canines, equines, felines, porcines, ungulates, largomorphs, and the like.

E. Stabilization of Vulnerable Plaques

As explained herein, heart disease, specifically coronary artery disease, is a major cause of death, disability, and healthcare expense in the United States and other industrialized countries. Until recently, most heart disease was considered to be primarily the result of a progressive increase of hard plaque in the coronary arteries. This atherosclerotic disease process of hard plaques leads to a critical narrowing (stenosis) of the affected coronary artery and produces anginal syndromes, known commonly as chest pain. The progression of the narrowing reduces blood flow, triggering the formation of a blood clot (thrombus). The clot may choke off the flow of oxygen-rich blood (ischemia) to heart muscles, causing a heart attack. Alternatively, the clot may break off and lodge in the vessel of another organ, such as the brain, resulting in a thrombotic stroke.

Within the past decade, however, evidence has emerged changing to some extent the paradigm of atherosclerosis, coronary artery disease, and heart attacks. While the buildup of hard plaque may produce angina and severe ischemia in the coronary arteries, new clinical data suggest that the rupture of vulnerable plaques, which are often non-occlusive, per se, causes the vast majority of heart attacks. The rate is estimated as high as 60-80 percent.

In many instances, vulnerable plaques do not impinge on the vessel lumen; rather, much like an abscess, they are ingrained within the arterial wall. The majority of vulnerable plaques include a lipid pool, smooth muscle (endothelial) cells, and a dense infiltrate of cholesterol filled macrophages/foam cells contained by a thin fibrous cap. The lipid pool is believed to be foamed as a result of pathological process involving low density lipoprotein (LDL), macrophages, and the inflammatory process. The macrophages oxidize the LDL, producing foam cells.

The macrophages, foam cells and associated endothelial cells release various substances, such as tumor necrosis factor, tissue factor, and matrix proteinases, which result in generalized cell necrosis and apoptosis, pro-coagulation, and weakening of the fibrous cap. The inflammation process may weaken the fibrous cap to the extent that sufficient mechanical stress, such as that produced by increased blood pressure, may result in rupture. The lipid core and other contents of the vulnerable plaque may then spill into the blood stream, thereby initiating a clotting cascade. The cascade produces a blood clot that potentially results in a heart attack and/or stroke. The process is exacerbated due to the release of collagen and plaque components (e.g., collagen and tissue factor), which enhance clotting upon their release.

It has been found that the polypeptides of the present invention can stabilize vulnerable plaques by reducing plaque lipid content through reverse cholesterol transport. Thus, in another embodiment, the present invention provides methods for stabilizing a vulnerable plaque in a blood vessel of a mammal by administering to the mammal (and, more preferably, a human), one or more of the polypeptides of this invention (or peptidomimetics of such polypeptides). A “vulnerable” plaque is generally defined as a lipid-rich plaque with a thinned fibrous cap lacking proper collagen and smooth muscle cell support. Again, the polypeptides of the present invention can reduce plaque lipid content, thereby stabilizing such “vulnerable” plaques.

In one embodiment, the mammal is a mammal diagnosed as having one or more vulnerable plaques. In this embodiment, a number of different diagnostic assays have been developed for the detection (e.g., diagnosis and localization) of vulnerable plaques, including temperature detection strategies, labeling strategies, imaging strategies (e.g., devices utilizing magnetic resonance, ultrasound, infra-red, fluorescence, visible light, radio waves, x-ray, etc.), general strategies for discriminating the vulnerable plaque from surround healthy vascular tissue and the like (see, e.g., U.S. Pat. Nos. 6,245,026, 6,475,159, 6,475,210 and 7,118,567). One strategy involves the measurement of temperature within a blood vessel. For example, vulnerable plaque tissue temperature is generally elevated compared to healthy vascular tissue. Measurement of this temperature discrepancy allows detection of the vulnerable plaque. Another detection strategy involves labeling vulnerable plaque with a marker. The marker can be a substance specific for a component and/or characteristic of the vulnerable plaque (such as C-reactive protein). For example, the marker may have an affinity for the vulnerable plaque, more so than for healthy tissue. Detection of the marker may thus allow detection of the vulnerable plaque. Alternatively, the marker may not necessarily have an affinity for the vulnerable plaque, but will simply change properties while associated with the vulnerable plaque. The property change may be detected and thus allow detection of the vulnerable plaque.

In another embodiment, the mammal is at risk of having one or more vulnerable plaques. In this embodiment, a clinical symptom has developed and/or a clinical event has occurred that leads one of skill in the art to believe that the mammal is at risk of having one or more vulnerable plaques.

In connection with the above methods of stabilizing a vulnerable plaque, the polypeptide(s) can be administered, as described herein, according to any of a number of standard methods including, but not limited to, injection, infusion, suppository, nasal spray, time-release implant, transdermal patch, orally and the like. In one particularly preferred embodiment, the polypeptide(s) is administered orally (e.g., as a syrup, capsule, tablet, etc.). In addition, the polypeptides (or peptidomimetics) of the present invention can be used alone or in combination with other known pharmaceutical agents for the treatment of dyslipidemia, hypercholesterolemia and inflammation to raise plasma HDL concentrations and/or to promote reverse cholesterol transport.

### VII. COMBINATION THERAPY

In some embodiments, the polypeptides or peptidomimetics of the present invention are administered in combination with one or more additional therapeutic agents for treating or preventing diseases and disorders associated with dyslipidemia, hypercholesterolemia and inflammation, such as cardiovascular disease, including atherosclerosis. For instance, in one embodiment, a polypeptide of the present invention is administered in conjunction with any of the standard treatments for atherosclerosis including, for example, statins (e.g., atorvastatin, lovastatin, pravastatin, simvastatin, fluvastatin, or rosuvastatin); a Nieman-Pick Cl-Like 1 sterol transporter channel inhibitor (e.g., Ezetimibe); bile acid binders (e.g., cholestyramine or colestipol); platelet clumping inhibitors (e.g., aspirin, ticlopidine, or clopidogrel); niacin/nicotinamide; PPAR activators; Vitamin E; surgical intervention (e.g., angioplasty, stents, stents, or endarterectomy); and lifestyle changes (e.g., low-fat diets, weight loss, and exercise).

More particularly, the polypeptides or peptidomimetics of the present invention can be used in combination, either as separate units or fixed combinations, with one or more of the following: an antibody which binds to an unwanted inflammatory molecule or cytokine such as interleukin-6, interleukin-8, granulocyte macrophage colony stimulating factor, and tumor necrosis factor-α; an enzyme inhibitor such as a protease inhibitor aprotinin or a cyclooxygenase inhibitor; an antibiotic such as amoxicillin, rifampicin, erythromycin; an antiviral agent such as acyclovir; a steroidal anti-inflammatory such as a glucocorticoid; a non-steroidal anti-inflammatory such as aspirin, ibuprofen or acetaminophen; or a non-inflammatory cytokine such as interleukin-4 or interleukin-10. Other cytokines and growth factors such as interferon-β, tumor necrosis factors, antiangiogenic factors, erythropoietins, thrombopoietins, interleukins, maturation factors, chemotactic protein, and their variants and derivatives that retain similar physiological activities may also be used as an additional therapeutic agents.

The polypeptides or peptidomimetics of the present invention can be used in combination with drugs commonly used to treat lipid disorders in, for example, diabetic patients. Such drugs include, but are not limited to, HMG-CoA reductase inhibitors, nicotinic acid, ezetimide, bile acid sequestrants, fibric acid derivatives, MTP inhibitor, ACAT inhibitor and CETP inhibitors. Examples of HMG-CoA reductase inhibitors include lovastatin, pravastatin, simvastatin, rosuvastatin, fluvastatin and atorvastatin. Examples of bile acid sequestrants include cholestyramine, colestipol and colesevelam. Examples of fibric acid derivatives include gemfibrozil and fenofibrate,

The polypeptides or peptidomimetics of the invention can also be used in combination with anti-hypertensive drugs, such as, for example, diuretics, β-blockers, cathepsin S inhibitors, methyldopa, α2-adrenergic agonists, guanadrel, reserpine, β-adrenergic receptor antagonists, α1-adrenergic receptor antagonists, hydralazine, minoxidil, calcium channel antagonists, ACE inhibitors and angiotensin II-receptor antagonists. Examples of β-blockers include acebutolol, bisoprolol, esmolol, propanolol, atenolol, labetalol, carvedilol and metoprolol. Examples of ACE inhibitors include captopril, enalapril, lisinopril, benazepril, fosinopril, ramipril, quinapril, perindopril, trandolapril and moexipril.

The polypeptides or peptidomimetics of the invention can also be used in combination with cardiovascular drugs such as calcium channel antagonists, β-adrenergic receptor antagonists and agonists, aldosterone antagonists, ACE inhibitors, angiotensin II receptor antagonists, nitrovasodilators, and cardiac glycosides. The polypeptides or peptidomimetics of the invention can also be used in combination with anti-inflammatory drugs such as H1-receptor antagonists, H2-receptor mediated agonists and antagonists, COX-2 inhibitors, NSAID, salicylates, acetaminophen, propionic acid derivatives, enolic cids, diaryl substituted fuanones, cyclooxygenase inhibitors, and bradykinin agonists and antagonists.

Other therapeutic agents suitable for use in combination with the polypeptides or peptidomimetics of the present invention are disclosed in U.S. Patent Application Publication No. 2005/0142180, which was published Jun. 30, 2005, the teachings of which are incorporated herein by reference.

The polypetide (or peptidomimetics thereof) and the additional therapeutic agent can be administered simultaneously or sequentially. For example, the polypeptide may be administered first, followed by the additional therapeutic agent. Alternatively, the additional therapeutic agent may be administered first, followed by the polypeptide of the invention. In some cases, the polypeptide of the invention and the additional therapeutic agent are administered in the same formulation. In other cases, the polypeptide and the additional therapeutic agent are administered in different formulations. When the polypeptide and the additional therapeutic agent are administered in different formulations, their administration may be simultaneous or sequential.

### VIII. PHARMACEUTICAL FORMULATIONS

In order to carry out the methods of the invention, one or more polypeptides of this invention or peptidomimetics thereof are administered to an individual diagnosed as having or at risk of having a disease or disorder associated with dyslipidemia, hypercholesterolemia and inflammation (e.g., to an individual diagnosed as having one or more symptoms of atherosclerosis, or as being at risk for atherosclerosis). The polypeptides or peptidomimetics thereof can be administered in their “native” form or, if desired, in the form of, for example, salts, esters, amides, prodrugs, derivatives, and the like, provided that the salt, ester, amide, prodrug or derivative is suitable pharmacologically, i.e., effective in the methods of the present invention.

In one embodiment of the methods described herein, the route of administration can be oral, intraperitoneal, transdermal, subcutaneous, by intravenous or intramuscular injection, by inhalation, topical, intralesional, infusion; liposome-mediated delivery; topical, intrathecal, gingival pocket, rectal, intrabronchial, nasal, transmucosal, intestinal, ocular or otic delivery, or any other methods known in the art as one skilled in the art may easily perceive. Other embodiments of the compositions of the invention incorporate particulate forms protective coatings, protease inhibitors or permeation enhancers for various routes of administration, including parenteral, pulmonary, nasal and oral. The pharmaceutical compositions can be administered in a variety of unit dosage forms depending upon the method/mode of administration. Suitable unit dosage forms include, but are not limited to, powders, tablets, pills, capsules, lozenges, suppositories, patches, nasal sprays, injectibles, implantable sustained-release formulations, etc.

As such, in another aspect, the present invention provides pharmaceutical compositions comprising a pharmaceutically effective amount of a polypeptide or peptidomimetic of the present invention and an acceptable carrier and/or excipients. A pharmaceutically acceptable carrier includes any solvents, dispersion media, or coatings that are physiologically compatible and that preferably does not interfere with or otherwise inhibit the activity of the polypeptide or peptidomimetic. Preferably, the carrier is suitable for intravenous, intramuscular, oral, intraperitoneal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include, but are not limited to, wetting agents, emulsifying agents, dispersing agents or preservatives which are particularly useful for preventing the growth or action of microorganisms. Various preservatives are well known and include, for example, phenol and ascorbic acid. One skilled in the art will appreciate that the choice of pharmaceutically acceptable carrier(s), including a physiologically acceptable compound depends, for example, on the route of administration of the polypeptide(s) or peptidomimetic(s) and on the particular physio-chemical characteristics of the polypeptide(s) or peptidomimetic(s).

In a preferred embodiment, the pharmaceutically acceptable carrier is physiological saline. Other pharmaceutically acceptable carriers and their formulations are well-known and generally described in, for example, Remington's Pharmaceutical Science (18^{th} Ed., ed. Gennaro, Mack Publishing Co., Easton, Pa., 1990). Various pharmaceutically acceptable excipients are well-known in the art and can be found in, for example, Handbook of Pharmaceutical Excipients (4^{th} ed., Ed. Rowe et al., Pharmaceutical Press, Washington, D.C.). Again, the pharmaceutical composition can be formulated as a solution, microemulsion, liposome, capsule, tablet, or other suitable form. The active component may be coated in a material to protect it from inactivation by the environment prior to reaching the target site of action.

In certain preferred embodiments, the polypeptides or peptidomimetics of this invention can be administered orally (e.g., via a tablet) or as an injectable in accordance with standard methods well known to those of skill in the art. In other preferred embodiments, the polypeptides or peptidomimetics can also be delivered through the skin using conventional transdermal drug delivery systems, i.e., transdermal “patches,” wherein the polypeptide(s) or peptidomimetic(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other preferred formulations for topical drug delivery include, but are not limited to, ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

In some embodiments, implanted devices (e.g., arterial and intravenous stents, including eluting stents, and catheters) are used to deliver the formulations comprising the polypeptides and peptidomimetics of the invention. For example, aqueous solutions comprising the polypeptides and peptidomimetics of the invention are administered directly through the stents and catheters. In some embodiments, the stents and catheters may be coated with formulations comprising the polypeptides and peptidomimetics described herein. In some embodiments, the polypeptides and peptidomimetics will be in time-release formulations an eluted from the stents. Suitable stents are described in, e.g., U.S. Pat. Nos. 6,827,735; 6,827,735; 6,827,732; 6,824,561; 6,821,549; 6,821,296; 6,821,291; 6,818,247; 6,818,016; 6,818,014; 6,818,013; 6,814,749; 6,811,566; 6,805,709; 6,805,707; 6,805,705; 6,805,704; 6,802,859; 6,802,857; 6,802,856; and 49 6,802,849. Suitable catheters are described in, e.g., U.S. Pat. Nos. 6,829,497; 6,827,798; 6,827,730; 6,827,703 ; 6,824,554; 6,824,553; 6,824,551; 6,824,532; and 6,819,951.

Unlike typical polypeptide formulations, the polypeptides of this invention comprising L-form or D-form amino acids can be administered, even orally, without protection against proteolysis by stomach acid, etc. Nevertheless, in certain embodiments, polypeptide delivery can be enhanced by the use of protective excipients. This is typically accomplished either by complexing the polypeptide with a composition to render it resistant to acidic and enzymatic hydrolysis, or by packaging the polypeptide in an appropriately resistant carrier such as a liposome. Means of protecting polypeptides for oral delivery are well known in the art (see, e.g., U.S. Pat. No. 5,391,377, which describes lipid compositions for oral delivery of therapeutic agents).

Elevated serum half-life can be maintained by the use of sustained-release polypeptide “packaging” systems. Such sustained release systems are well known to those of skill in the art. In one preferred embodiment, the ProLease biodegradable microsphere delivery system for proteins and polypeptides is used (Tracy, Biotechnol. Prog., 14:108 (1998); Johnson et al., Nature Med., 2:795 (1996); Herbert et al., Pharmaceut. Res., 15:357 (1998)), which involves the use of a dry powder composed of biodegradable polymeric microspheres containing the polypeptide in a polymer matrix that can be compounded as a dry formulation with or without other agents.

The ProLease microsphere fabrication process was designed to achieve a high polypeptide encapsulation efficiency while maintaining protein integrity. The process consists of (i) preparation of freeze-dried protein particles from bulk polypeptide by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the polypeptide, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the polypeptide is maintained in the solid state in the absence of water, thus minimizing water-induced conformational mobility of the polypeptide, preventing polypeptide degradation reactions that include water as a reactant, and avoiding organic-aqueous interfaces where polypeptides may undergo denaturation. A preferred process uses solvents in which most polypeptides are insoluble, thus yielding high encapsulation efficiencies (e.g., greater than 95%).

In another embodiment, one or more components of the solution can be provided as a “concentrate,” e.g., in a storage container (e.g., in a premeasured volume) ready for dilution, or in a soluble capsule ready for addition to a volume of water.

In certain embodiments of the present invention, the pharmaceutical compositions are sustained release formulations. Polypeptides or peptidomimetics of the present invention may be admixed with biologically compatible polymers or matrices which control the release rate of the copolymers into the immediate environment. Controlled or sustained release compositions include formulation in lipophilic depots (e.g., fatty acids, waxes, oils). Also contemplated by the invention are particulate compositions coated with polymers (e.g., poloxamers or poloxamines). Other embodiments of the compositions of the invention incorporate particulate forms, protective coatings, protease inhibitors or permeation enhancers for various routes of administration, including parenteral, pulmonary, nasal and oral. Acceptable carriers include carboxymethyl cellulose (CMC) and modified CMC.

The pharmaceutical composition of the present invention is preferably sterile and non-pyrogenic at the time of delivery, and is preferably stable under the conditions of manufacture and storage. These pharmaceutical compositions can be sterilized by conventional, well known sterilization techniques.

In therapeutic applications, the compositions of this invention are administered to an individual diagnosed as having or at risk of having a disease or disorder associated with dyslipidemia, hypercholesterolemia and inflammation (and, in preferred embodiments, to an individual diagnosed as having one or more symptoms of atherosclerosis or as being at risk for atherosclerosis) in an amount sufficient to cure or at least partially prevent or arrest the disease, condition and/or its complications. An amount adequate to accomplish this is defined as a “therapeutically effective dose.” Amounts effective for this use will depend upon the severity of the disease and the general state of the patient's health. Single or multiple administrations of the compositions can be administered depending on the dosage and frequency as required and tolerated by the patient. In any event, the composition should provide a sufficient quantity of the active agents, i.e., polypeptides or peptidomimetics, of the formulations of this invention to effectively treat (ameliorate one or more symptoms) the individual or patient.

The concentration of polypeptide or peptidomimetic can vary widely, and will be selected primarily based on fluid volumes, viscosities, body weight, circulating plasma levels of the polypeptide, polypeptide toxicities, progression of the disease (e.g., atherosclerosis), the production of antibodies that specifically bind to the polypeptide, and the like in accordance with the particular mode of administration selected and the patient's needs. Typically, the dose equivalent of a polypeptide or peptidomimetic is from about 0.1 to about 50 mg per kg, preferably from about 1 to about 25 mg per kg, most preferably from about 1 to about 20 mg per kg body weight. It will be appreciated that such dosages may be varied to optimize a therapeutic regimen in a particular subject or group of subjects.

For administration, polypeptides of the present invention can be administered at a rate determined by the LD50 of the polypeptide, and the side-effects of the polypeptide at various concentrations, as applied to the mass and overall health of the patient. Administration can be accomplished via single or divided doses, e.g., doses administered on a regular basis (e.g., daily) for a period of time (e.g., 2, 3, 4, 5, 6, days or 1-3 weeks or more).

As explained herein, the polypeptides or peptidomimetics of the present invention can be modified in a number of different ways. For instance, the polypeptides can be modified so that the R-groups on the constituent amino acids and/or the terminal amino acids are blocked, i.e., protected, by a protecting group. It has been found that blockage, particularly of the amino and/or carboxy termini, can greatly improve oral delivery and significantly increases serum half-life. In addition, to enhance delivery and/or biological acitivites in vivo, salts, esters, amides, prodrugs and other derivatives of the polypeptides or peptidomimetics of the present invention can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

For example, acid addition salts are prepared from the free base using conventional methodology, which typically involves reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the polypeptides described herein are halide salts, such as may be prepared using hydrochloric or hydrobromic acids. Conversely, preparation of basic salts of the polypeptides or peptidomimetics of the present invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., sodium salts and copper salts.

Preparation of esters typically involves functionalization of hydroxyl and/or carboxyl groups that may be present within the polypeptides or peptidomimetics of the present invention. The esters are typically acyl-substituted derivatives of free alcohol groups, i.e., moieties that are derived from carboxylic acids of the formula RCOOH, wherein R is alkyl and, preferably, lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

The foregoing formulations and administration methods are clearly intended to be illustrative and not limiting in any way. It will be appreciated that, using the teaching provided herein, other suitable formulations and modes of administration can be readily devised.

### IX. LIPID-BASED FORMULATIONS

In another aspect, the polypeptides and peptidomimetics of the present invention are preferably administered in conjunction with one or more lipids. The lipids can be formulated as an excipient to protect and/or enhance transport/uptake of the polypeptides or peptidomimetics or they can be administered separately.

The lipids can be formulated into liposomes, nanocapsules, microparticles, microspheres, lipids particles, lipid vesicles and the like. Such lipid formulations can be used to encapsulated the polypeptides and peptidomimetics of the present invention and/or they can be simply complexed/admixed with such polypeptides and peptidomimetics. Those of skill in the art will know how to use such lipid formulations to either encapsulate or complex the polypeptides or peptidomimetics of the present invention. For instance, the formation and use of liposomes is generally known to those of skill in the art. Recently, liposomes were developed with improved serum stability and circulation half-times (see, U.S. Pat. No. 5,741,516). Further, various methods of liposome and liposome-like preparations as potential drug carriers have been reviewed (see, U.S. Pat. Nos. 5,567,434; 5,552,157; 5,565,213; 5,738,868 and 5,795,587).

In one embodiment, the polypeptides or peptidomimetics of the present invention are complexed with a lipid, such as a phospholipid (e.g., 1-palmitoyl-2-oleoyl-sn-glycerol-phosphatidylcholine (“POPC”) in a manner similar to that disclosed in U.S. Patent Application Publication No. 2005/0142180, which was published Jun. 30, 2005, the teachings of which are incorporated herein by reference. It has surprisingly been found that when the polypeptides and peptidomimetics of the present invention are complexed with, for example, POPC at ratios ranging from about 1:0.5 to about 1:5 (polypeptide:POPC), distinct lipid-polypeptide particles are formed having sizes of between about 5 and about 20 nm, which result in a significantly enhanced capacity, i.e., ability, to efflux cholesterol from cells.

As such, the present invention provides polypeptide-lipid complexes (or, alternatively, peptidomimetic-lipid complexes) having an increased ability to efflux cholesterol from cells. Typically, the lipid is mixed with the polypeptide prior to administration. The polypeptides of the present invention and lipids can be mixed in an aqueous solution in appropriate ratios and can be complexed by methods known in the art, including, but not limited to, freeze-drying, detergent solubilization followed by dialysis, microfluidization, sonication, and homogenization. Complex efficiency can be optimized, for example, by varying pressure, ultrasonic frequency or detergent concentration. An example of a detergent commonly used to prepare polypeptide-lipid complexes is sodium cholate.

In certain embodiments, the polypeptide-lipid (e.g., phospholipids) complex can be in solution with an appropriate pharmaceutical diluent or carrier. In other embodiments, freeze-dried or lyophilized preparations of the polypeptide-lipid complexes can be hydrated or reconstituted with an appropriate pharmaceutical diluent prior to administration. In another embodiment, the polypeptide-lipid complexes can be frozen preparations that are thawed until a homogenous solution is achieved prior to administration to a subject in need thereof.

The lipid can be any suitable lipid known to those of skill in the art. In one embodiment, non-phosphorus containing lipids can be used, including stearylamine, dodecylamine, acetyl palmitate, (1,3)-D-mannosyl-(1,3)digly-ceride, aminophenylglycoside, 3-cholesteryl-6′-(glycosylthio)hexyl ether glycolipids, N-(2,3-di(9-(Z)-octadecenyloxy))-prop-1-yl-N,N,N-trimethylammonium chloride and fatty acid amides.

In another embodiment, a phospholipids or a mixture of phospholipids is used. Suitable phospholipids include, but are not limited to, can be a small alkyl chain phospholipid, phosphatidylcholine, egg phosphatidylcholine, soybean phosphatidylcholine, dipalmitoylphosphatidylcholine, soy phosphatidylglycerol, egg phosphatidylglycerol, distearoylphosphatidylgly-cerol, dimyristoylphosphatidylcholine, distearoylphosphatidylcholine, dilaurylphosphatidylcholine, 1-myristoyl-2-palmitoylphosphatidylcholine, 1-palmitoyl-2-myristoylphosphatidylcholine, 1-palmitoyl-2-stearoylphospha-tidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, phosphatidylglycerol, phosphatidic acid, lysolecithin, lysophosphatidylethanolamine, cephalin, cardiolipin, dicetylphosphate, distearoyl-phosphatidylethanolamine and cholesterol and its derivatives. Similarly, the phospholipid can be a derivative or analogue of any of the foregoing phospholipids or, again, a mixture of two or more of any of the foregoing phospholipids. Such phospholipids can be obtained from commercial sources, natural sources or by synthetic or semi-synthetic means known to those of skill in the art.

In preferred embodiments, the polypeptide-lipid complex is a polypeptide-phospholipid-complex. In a more preferred embodiment, the lipid is 1-palmitoyl-2-oleoyl phosphatidylcholine (“POPC”) or (“1-palmitoyl-2-oleoyl-sn-glycero-3-phosphocholine”).

It will be readily apparent to those of skill in the art that the complex comprising a polypeptide of the present invention and a lipid, preferably a phospholipids, can comprise any amount of lipid and any amount of the polypeptide, provided the complex is effective to mediate cholesterol efflux and, in turn, to treat diseases or symptoms associate therewith. As previously mentioned, it has surprisingly been found that when the polypeptides of the present invention are complexed with, for example, POPC at ratios ranging from about 1:0.5 to about 1:5 (polypeptide:POPC), distinct lipid-polypeptide particles are formed having sizes of between about 5 and about 20 nm, which result in a significantly enhanced capacity, i.e., ability, to efflux cholesterol from cells. However, the polypeptide-lipid complexes of the present invention can comprise complexes with other ratios of phospholipid to polypeptide, such as about 100:1, about 10:1, about 5:1, about 3:1, about 2:1, about 1:1, about 1:2, about 1:3, about 1:5, about 1:10 and about 1:100 (wt of polypeptide/wt of lipid).

The polypeptide-lipid complexes of the present invention can be made by any method known to one of skill in the art. In some cases, it is desirable to mix the lipid and the polypeptide prior to administration. Lipids can be in solution or in the form of liposomes or emulsions formed using standard techniques, such as homogenization, sonication or extrusion. Sonication is generally performed with a tip sonifier, such as a Branson tip sonifier, in an ice bath. Typically, the suspension is subjected to several sonication cycles. Extrusion can be carried out by biomembrane extruders, such as the Lipex Biomembrane Extruder™ (Lipex Biomembrane Extruder, Inc. Vancouver, Canada). Defined pore size in the extrusion filters can generate unilamellar liposomal vesicles of specific sizes. The liposomes can also be formed by extrusion through an asymmetric ceramic filter, such as a Ceraflow Microfilter™, which is commercially available from the Norton Company, Worcester, Mass., or through a polycarbonate filter or other types of polymerized materials (i.e., plastics) known to those of skill in the art.

As previously mentioned, the polypeptide-lipid complexes of the present invention can be prepared in a variety of forms including, but not limited to, vesicles, liposomes or proteoliposomes. A variety of methods well known to those skilled in the art can be used to prepare the polypeptide-lipid complexes. A number of available techniques for preparing liposomes or proteoliposomes can be used. For example, a polypeptide of the present invention (e.g., a polypeptide of SEQ ID NOS:1-3) can be co-sonicated (using a bath or probe sonicator) with the appropriate lipid to form the polypeptide-lipid complexes. In certain embodiments, the polypeptide can be combined with preformed lipid vesicles resulting in the spontaneous formation of an polyeptide-lipid complex. In another embodiment, the polypeptide-lipid complex can also be made by a detergent dialysis method. In this method, a mixture of the polypeptide, lipid and a detergent, such as sodium cholate, can be dialyzed to remove the detergent and reconstituted to make the polypeptide-lipid complexes (see, e.g., Jonas et al., Methods Enzymol., 128:553-82 (1986)).

In other embodiments, the polypeptide-lipid complexes can be made by co-lyophilization as described in U.S. Pat. Nos. 6,287,590 and 6,455,088, the teachings of both of which are hereby incorporated by reference in their entirety. Other methods are disclosed in, for example, U.S. Pat. Nos. 6,004,925, 6,037,323 and 6,046,166, the teachings of all of which are incorporated herein by reference in their entireties. Other methods of preparing polypeptide-lipid complexes will be apparent to those of skill in the art.

In one preferred embodiment, the polypeptide-lipid complexes can be made by homogenization.

### X. NUCLEIC ACIDS AND GENE THERAPY

In another embodiment, the present invention provides isolated nucleic acids encoding the polypeptides disclosed herein, expression vectors comprising the nucleic acids, and host cells comprising the expression vectors. More particularly, the present invention provides isolated nucleic acids encoding the polypeptides of the present invention having cholesterol efflux activities similar to full-length apolipoproteins, on a per molecule basis, and having high selectivity for ABAC1 in a manner similar to full-length apolipoproteins, the polypeptides including, but not limited to, the polypeptides having an amino acid sequence comprising SEQ ID NOS:1-3.

In certain embodiments, nucleic acids encoding the polypeptides of the invention are used for transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acids, under the control of a promoter, then express a polypeptide of the present invention, thereby mitigating the effects of a disease associated with dyslipidemia, hypercholesterolemia and inflammation.

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies (for a review of gene therapy procedures, see Anderson, Science, 256:808-813 (1992); Nabel et al., TIBTECH, 11:211-217 (1993); Mitani et al., TIBTECH, 11:162-166 (1993); Mulligan, Science, 926-932 (1993); Dillon, TIBTECH, 11:167-175 (1993); Miller, Nature, 357:455-460 (1992); Van Brunt, Biotechnology, 6(10):1149-1154 (1998); Vigne, Restorative Neurology and Neuroscience, 8:35-36 (1995); Kremer et al., British Medical Bulletin, 51(1):31-44 (1995); Haddada et al., in Current Topics in Microbiology and Immunology (Doerfler & Bohm eds., 1995); and Yu et al., Gene Therapy, 1:13-26 (1994)).

For delivery of nucleic acids, viral vectors may be used. Suitable vectors include, for example, herpes simplex virus vectors as described in Lilley et al., Curr. Gene Ther., 1(4):339-58 (2001), alphavirus DNA and particle replicons as decribed in e.g., Polo et al., Dev. Biol. (Basel), 104:181-5 (2000), Epstein-Barr virus (EBV)-based plasmid vectors as described in, e.g., Mazda, Curr. Gene Ther., 2(3):379-92 (2002), EBV replicon vector systems as described in e.g., Otomo et al., J. Gene Med., 3(4):345-52 (2001), adeno-virus associated viruses from rhesus monkeys as described in e.g., Gao et al., PNAS USA., 99(18):11854 (2002), adenoviral and adeno-associated viral vectors as described in, e.g., Nicklin et al., Curr. Gene Ther., 2(3):273-93 (2002). Other suitable adeno-associated virus (AAV) vector systems can be readily constructed using techniques well known in the art (see, e.g., U.S. Pat. Nos. 5,173,414 and 5,139,941; PCT Publication Nos. WO 92/01070 and WO 93/03769; Lebkowski et al., Mol. Cell. Biol., 8:3988-3996 (1988); Vincent et al. (1990) Vaccines 90 (Cold Spring Harbor Laboratory Press); Carter, Current Opinion in Biotechnology 3:533-539 (1992); Muzyczka, Current Topics in Microbiol. and Immunol., 158:97-129 (1992); Kotin, Human Gene Therapy, 5:793-801 (1994); Shelling et al., Gene Therapy, 1:165-169 (1994); and Zhou et al., J. Exp. Med., 179:1867-1875 (1994)). Additional suitable vectors include E1B gene-attenuated replicating adenoviruses described in, e.g., Kim et al., Cancer Gene Ther., 9(9):725-36 (2002) and nonreplicating adenovirus vectors described in e.g., Pascual et al., J. Immunol., 160(9):4465-72 (1998) Exemplary vectors can be constructed as disclosed by Okayama et al., Mol. Cell. Biol., 3:280 (1983).

Molecular conjugate vectors, such as the adenovirus chimeric vectors described in

Michael et al., J. Biol. Chem., 268:6866-6869 (1993) and Wagner et al., Proc. Natl. Acad. Sci. USA, 89:6099-6103 (1992), can also be used for gene delivery according to the methods of the invention.

In one illustrative embodiment, retroviruses provide a convenient and effective platform for gene delivery systems. A selected nucleotide sequence encoding a polypeptide of the invention is inserted into a vector and packaged in retroviral particles using techniques known in the art. The recombinant virus can then be isolated and delivered to a subject. Suitable vectors include lentiviral vectors as described in e.g., Scherr et al., Curr. Gene Ther., 2(1):45-55 (2002). Additional illustrative retroviral systems have been described (e.g., U.S. Pat. No. 5,219,740; Miller et al., BioTechniques, 7:980-990 (1989); Miller, Human Gene Therapy, 1:5-14 (1990); Scarpa et al., Virology, 180:849-852 (1991); Burns et al., Proc. Natl. Acad. Sci. USA, 90:8033-8037 (1993); and Boris-Lawrie et al., Curr. Opin. Genet. Develop., 3:102-109 (1993).

Other known viral-based delivery systems are described in, e.g., Fisher-Hoch et al., Proc. Natl. Acad. Sci. USA, 86:317-321 (1989); Flexner et al., Ann. N.Y. Acad. Sci., 569:86-103 (1989); Flexner et al., Vaccine, 8:17-21 (1990); U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques, 6:616-627 (1988); Rosenfeld et al., Science, 252:431-434 (1991); Kolls et al., Proc. Natl. Acad. Sci. USA, 91:215-219 (1994); Kass-Eisler et al., Proc. Natl. Acad. Sci. USA, 90:11498-11502 (1993); Guzman et al., Circulation, 88:2838-2848 (1993); Guzman et al., Cir. Res., 73:1202-1207 (1993); and Lotze et al., Cancer Gene Ther., 9(8):692-9 (2002).

### XI. KITS

In another aspect, the present invention provides kits for the treatment, i.e., amelioration, or prevention of a disease or disorder, i.e., condition, associated with dyslipidemia, hypercholesterolemia and inflammation. In a preferred embodiment, the present invention provides kits for the treatment, i.e., amelioration, of one or more symptoms of atherosclerosis or for the prophylactic treatment of a subject (e.g., human or animal) at risk for atherosclerosis. The kits preferably comprise a container containing one or more of the polypeptides (or peptidomimetics) of this invention. The polypeptide or peptidomimetic can be provided in a unit dosage formulation (e.g., tablet, caplet, patch, suppository, etc.) and/or can be optionally combined with one or more pharmaceutically acceptable excipients.

The kit can, optionally, further comprise one or more other agents used in the treatment of a disease or condition associated with dyslipidemia, hypercholesterolemia and inflammation (such as heart disease and/or atherosclerosis). Such agents include, but are not limited to, those set forth above in connection with the section on “Combination Therapy.” For instance, in certain embodiments, the kit can include beta blockers, vasodilators, aspirin, statins, ace inhibitors or ace receptor inhibitors (ARBs) and the like.

In addition, the kits can optionally include labeling and/or instructional materials providing directions (i.e., protocols) for the practice of the methods or use of the “therapeutics” or “prophylactics” of this invention. Preferred instructional materials describe the use of one or more polypeptides or peptidomimetics of this invention, for example, to mitigate one or more symptoms of atherosclerosis and/or to prevent the onset or increase of one or more of such symptoms in an individual at risk for atherosclerosis. The instructional materials can also, optionally, teach preferred dosages/therapeutic regiment, counter indications and the like.

In some embodiments, the invention provides a kit comprising an oxidation resistant polypeptide and an oxidation sensitive counterpart polypeptide, e.g., SEQ ID NO:3 and SEQ ID NO:4, for diagnostic purposes.

While the instructional materials typically comprise written or printed materials, they are not limited to such. Any medium capable of storing such instructions and communicating them to an end user is contemplated by this invention. Such media include, but are not limited to, electronic storage media (e.g., magnetic discs, tapes, cartridges, chips, etc.), optical media (e.g., CD ROM), and the like. Such media may include addresses to internet sites that provide such instructional materials.

The invention will be described in greater detail by way of specific examples. The following examples are offered for illustrative purposes, and are not intended to limit the invention in any manner. Those of skill in the art will readily recognize a variety of non-critical parameters that can be changed or modified to yield essentially the same results.

### XII. EXAMPLES

### Example 1

The following example shows sequence substitutions in an exemplar polypeptide that were made to increase oxidation resistance.

The peptide ATI-5261 was used as a starting peptide to make substitutions. The sequence of ATI-5261 is: EVRSKLEEWFAAFREFAEEFLARLKS. Two peptides were generated, one of these is an analog having arginine substituted for lysine at positions 5 and 25. A second peptide included a change to a tryptophan residue at position 9:

Peptide K5, 25→R analog of ATI-5261
EVRSRLEEWFAAFREFAEEFLARLRS
Peptide K5, 25→R, W9→L (or F) analog
EVRSRLEELFAAFREFAEEFLARLRS

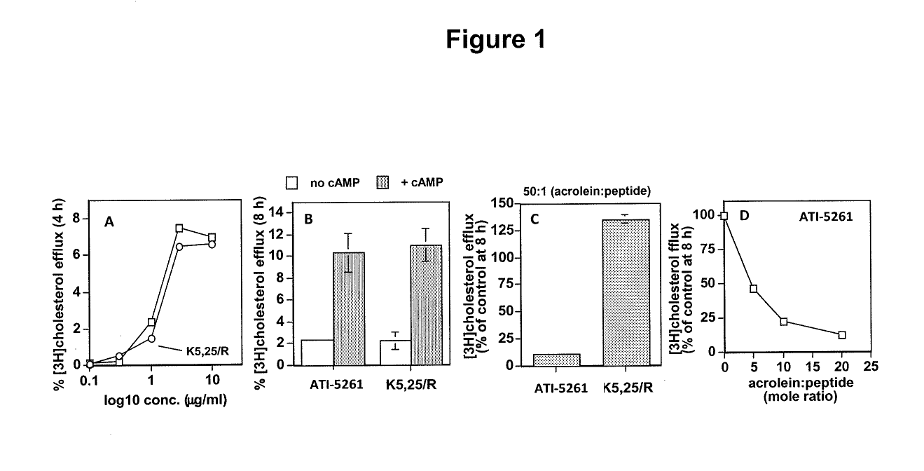

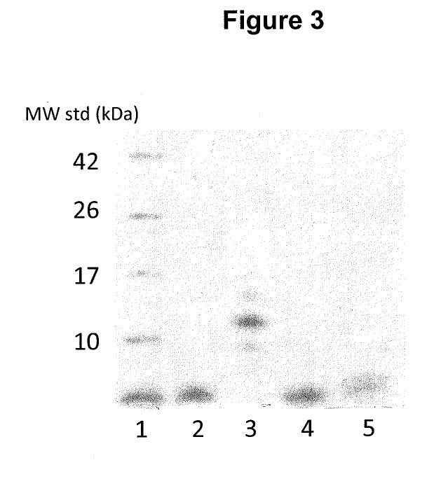

The residues that are different from parent ATI-5261 are underlined. The peptides were tested for sensitivity to oxidation (FIG. 1). The results show that K→R peptides are highly resistant to aldehydic oxidation products, retaining high colesterol efflux activity upon exposure to excess molar acrolein The α-helical content (secondary structure) of K5,25→R peptide was lower vs. the parent ATI-5261 peptide; however, α-helical content increased with acrolein exposure. The cholesterol efflux activity of K5,25→R peptide increased upon exposure to acrolein. In contrast, acrolein treatment of ATI-5261 produced a marked decrease in cholesterol efflux activity, reduced α-helical content and resulted in substantial cross-linking of the peptide (FIG. 3).
α-helical content: ATI-5261 Biosyn: 72.7%
α-helical content ATI-5261 +Acrolein: 48.3%
α-helical content K5,25/R: 56.8%
α-helical content K5,25/R +Acrolein: 72.6%

### Example 2

### Other Oxidation-Resistnat (Ox-R) and Oxidation-Susceptivel (Ox-S) Forms of ATI-5261

The following provides an example of motifs of oxidation sensitive and resistant peptides.

| | |
|---|---|
| EXXXKXXE YXXXK | (hypersensitive motif configuration) |
| EVRSKLEEWFAAFREFAEEFYARLKS | Ox-S peptide |
| EVRSRLEELFAAFREFAEEFYARLRS | OX-R peptide |
| ELRSKLEEWFAAFREFAEEFYARLKS | Ox-S peptide (with V2→L substitution) |
| ELVRSRLEELFAAFREFAEEFYARLRS | OX-R peptide (with V2→L substitution) |

The oxidation susceptible residues (within motifs) are underlined. Amino acid residues in bold confer resistance to oxidation. W and Y are considered major targets of myeloperoxidase (MPO) produced oxidatants. MPO also generates acrolein, which targets lysine residues. V2→L substitutions were used to improve cholesterol efflux efficiency of peptides. Tyrosine (Y) was used in constructing the YXXXK motif, which is slightly less polar than L21, which is normally present in ATI-5261.

Differential cholesterol efflux/RCT response between Ox-R and Ox-S is useful as a biomarker to assess disease susceptibility, vulnerable plaque and/or response to therapies.

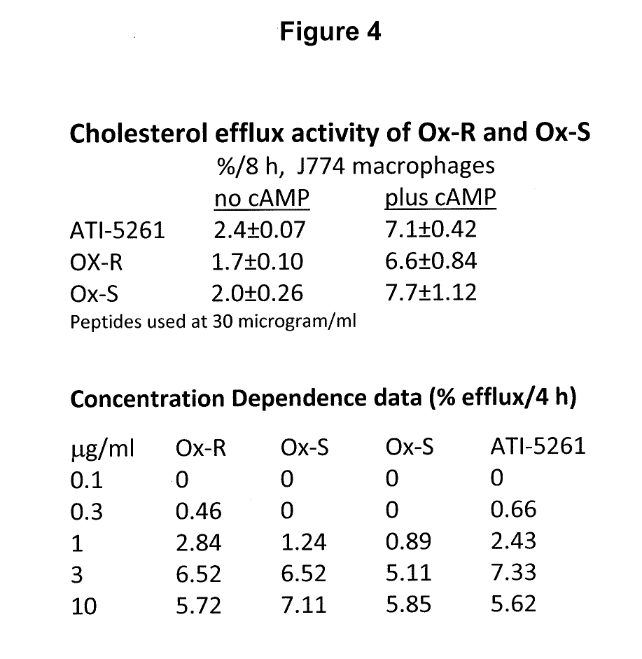

The table presented in FIG. 4 shows the cholesterol efflux activity of Ox-R and Ox-S:

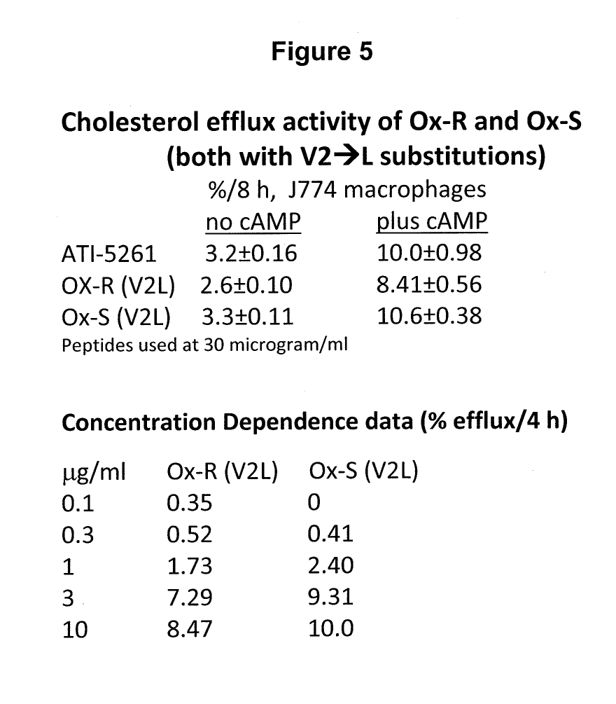

The table presented in FIG. 5 shows the cholesterol efflux activity of Ox-R and Ox-S peptides having a V2→L substitution.

Based on the analysis, single R→Q substitutions (which remove positive charges) at positions 3, 14, and 23 does not alter cholesterol efflux. Reversing the sequence of ATI-5261 inverted the pattern of positive- (polar face) and hydrophobic-residues (apolar face), but neither class A structure nor efflux activity were affected. Furthermore, leucine (L) could substitute for tryptophan (W), in contrast to findings with apoA-I. Thus, no specific configuration of hydrophobic or positively charged residues was required for mediating ABCA1 cholesterol efflux. Molar acrolein:peptide (K5,25 analog) ratios of 5:1, 10:1, 20:1 dramatically and dose-dependently reduced cholesterol efflux activity by 50±5, 70±3, 93±3%, respectively, indicating that lysine residues indirectly modulated efflux activity via susceptibility to aldehyde. In contrast, cholesterol efflux activity of the K→R peptide was ˜70% augmented by acrolein.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated herein by reference for all purposes.

1. A method of synthesizing an oxidation-resistant polypeptide that has cholesterol efflux activity, the method comprising synthesizing a peptide of less than about 100 amino acids in length that comprises an amphipathic alpha helix of 18 to 40 amino acids in length, wherein
the polar face comprises at least three acidic amino acids within the alpha helix secondary structure;
a positively charged residue at the lipid/water interface of the amphipathic alpha helix is an arginine; and
the peptide does not include tryptophan, tyrosine, cysteine, methionine, or lysine residues.

2. The method of claim 1, wherein the amphipathic alpha helix comprises the following residues:
(SEQ ID NO: 1)
X₁X₂X₃X₄RX₆X₇X₈(L/F)X₁₀X₁₁X₁₂X₁₃X₁₄X₁₅X₁₆X₁₇X₁₈X₁₉X₂₀X₂₁X₂₂X₂₃X₂₄ RX₂₆
wherein:
X₁, X₇, X₈, X₁₅, X₁₈ and X₁₉ are amino acids independently selected from the group consisting of E and D;
X₂, X₆, X₁₀, X₁₂, X₁₃, X₁₆, X_{1 7}, X₂₀, X₂₁ and X₂₄ are amino acids independently selected from the group consisting of A, V, L, I, or F;
X₃, X₁₄ and X₂₃ are amino acids independently selected from the group consisting of R, A, V, L, I, F, G, S, T, N and Q, wherein at least two of X₃, X₁₄ and X₂₃ are R; and
X₄, X₁₁, X₂₂ and X₂₆ are amino acids independently selected from the group consisting of T, S, G, and A.

3. The method of claim 2, wherein X₂, X₆, X₁₀, X₁₂, X₁₃, X₁₆, X₁₇, X₂₀, X₂₁ and X₂₄ are amino acids independently selected from the group consisting of A, V, I, L, and F.

4. The method of claim 3, wherein X₂ is V or L.

5. The method of claim 3, wherein X₂, X₆, X₁₀, X₁₂, X₁₃, X₁₆, X₁₇, X₂₀, X₂₁ and X₂₄ are amino acids independently selected from the group consisting of L and F.

6. The method of claim 1, wherein the residue at position 9 is L.

7. The method of claim 1, wherein X₄, X₁₁, and X₂₂ are A.

8. The method of claim 1, wherein X₃, X₁₄ and X₂₃ are R.

9. The method of claim 1, wherein the amphipathic alpha helix comprises at least 80% identity, or at least 90% identity, to E(L/V)RSRLEE(L/F)FAAFREFAEEFLARLRS (SEQ ID NO:2), the residue at position 5 and 25 is R; and the residue at position 9 is L or F.

10.-11. (canceled)

12. A polypeptide synthesized in accordance with the method of claim 1.

13. The polypeptide of claim 12, wherein the polypeptide has increased cholesterol efflux activity relative to an oxidation-sensitive analog.

14. (canceled)

15. The polypeptide of claim 12, wherein the polypeptide comprises a protecting group is coupled to the amino or carboxy terminus.

16.-19. (canceled)

20. The of claim 12, wherein all enantiomeric amino acids are “D” amino acids.

21.-22. (canceled)

23. A peptidomimetic of a peptide of claim 12 wherein the peptidomimetic is a retro-inverso analog or a retro-enantio analog.

24. A composition comprising a polypeptide of claim 12; and a pharmaceutically acceptable carrier.

25.-29. (canceled)

30. A method for mediating cholesterol efflux in a mammal, said method comprising administering to said mammal a polypeptide of claim 12, whereby cholesterol efflux is mediated.

31.-33. (canceled)

34. A detectable affinity ligand comprising an isolated polypeptide in accordance with claim 12 directly or indirectly linked to a detectable moiety.

35. (canceled)

36. A kit comprising a peptide of claim 12; and an oxidation-sensitive analog of the peptide.

37. A method of evaluating cholesterol efflux in a mammal, the method comprising administering a peptide of claim 12.

38. The method of claim 37, further comprising administering an oxidation-sensitive analog of the peptide.
